(19) United States
(12) Patent Application Publication
(10) Pub. No.: US 2009/0274758 A1
(43) Pub. Date: Nov. 5, 2009

(54) Solid Composition for Intra-Oral Delivery of Insulin

(75) Inventors: Adel Pinhasi, Holon (IL); Mila Gomberg, Jerusalem (IL)

Correspondence Address: FOLEY AND LARDNER LLP;SUITE 500, 3000 K STREET NW, WASHINGTON, DC, 20007, US

(73) Assignee: DEXCEL PHARMA TECHNOLOGIES LTD., JERUSALEM (IL)

(21) Appl. No.: 11/887,653

(22) PCT Filed: Mar. 27, 2006
(86) PCT No.: PCT/IL2006/000381

§ 371 Date: Apr. 8, 2008

Related U.S. Application Data
(60) Provisional application No. 60/666,581, filed on Mar. 31, 2005.

(51) Int. Cl. A61K 9/10; A61K 38/28; A61P 3/10

(52) U.S. Cl. 424/484; 514/3; 424/486; 424/487

(57) ABSTRACT

The invention provides a solid composition for intra-oral delivery of insulin, comprising; insulin; a hydrophilic polymer matrix; and a phospholipid, providing insulin bioavailability of at least 5%.

The present invention relates to a solid composition for intra-oral delivery of insulin, and to a drug delivery system. The term intra-oral as used herein is intended to include delivery to the oral cavity, buccal, lingual and sublingual areas The invention is based on a new delivery system consisting of a mixture of a hydrophilic (water soluble, swellable) polymer, carefully chosen lipids, insulin, and optionally surfactant, preservative, antioxidant, stabilizers, flavors and sweeteners. The delivery system is preferably a bioadhesive system which is adhered to a soft tissue in the buccal, sublingual or other oral cavity areas to release insulin locally to be absorbed by mucosa for systemic absorption. The hydration occurs, upon exposing the system to the oral cavity liquid, which hydration is responsible for adhesion. Hydration of the system may simultaneously result in dissolution of the polymer and spontaneous arrangement of the lipid component into bilayer liposomes (vesicles), and/or micelles, lamellar structures (single or multilamellar) and/or emulsion structure and or any other liquid crystalline structures in situ. In this manner the insulin dose or a part of it, can be entrapped into the liposomes (vesicles) or other lipid arrangements. The absorption of insulin into the blood system can thereby mainly occur through intra-oral mucosa. A high oral bioavailability of insulin (more than 10%)using such a device is achieved.

### BACKGROUND OF THE INVENTION

A well-known problem with the administration of insulin is that it is susceptible to enzymatic degradation when administrated orally. For this reason, parenteral administration has been the most widely used method. However, administration by injection is both inconvenient and unpleasant for the patient, particularly because of the fact that injections must be repeated regularly over protracted periods. To avoid the discomfort of insulin injections, several noninjectable (nonparenteral) formulations of insulin have been studied.

A significant limitation to nonparenteral administration of insulin is that it is poorly absorbed across the mucosal membranes which line the exposed surfaces of the oral, rectal, and vaginal orifices, the cornea of the eye, and the gut, thus the bioavailability of insulin after nonparenteral administration to mucosal surfaces often is very low.

The oral cavity is the first site that an orally delivered drug encounters. It is characterized by a pH that is nearly neutral (6 to 7.5) and a relatively small surface area for drug absorption. The sublingual mucosa are endowed with a large blood flow and therefore offer an opportunity for drug absorption as do the buccal membranes (the gums). The residence time of a delivery system in the oral cavity is usually short, several seconds for a tablet that is being swallowed to several minutes for a lozenge that is being sucked. Small tablets can be held under the tongue for short periods of time to allow immediate drug delivery (e.g. Nitroglycerine tablets for vasodialation). Current research for delivery of systemic drugs through the oral cavity is mainly concerned with buccal delivery. Polymeric adhesives are used to affix the tablet to the gums through which the drug can diffuse over several hours. Targeting drugs for local treatment of oral cavity symptoms can be achieved by similar means. Films can also be used to deliver drugs to the oral cavity as will be described later.

To date, a wide variety of polypeptide drugs have been evaluated for buccal delivery. Buccal delivery of peptides and proteins has potential advantages over other available routes. It avoids degradation by gastrointestinal enzymes and first-pass hepatic metabolism. Buccal delivery has high patient compliance and excellent accessibility, and self-placement of a dosage form is possible. Because of the natural function (i.e. to line and protect the inner surface of the cheek) of the buccal mucosa, it is less sensitive to irritation and damage than the other absorptive mucosa. Furthermore, there are fewer proteolytic enzymes at work as compared with oral administration to the gastrointestinal tract and in addition, the buccal mucosa is highly vascularized.

Although many penetration enhancers have been tested, so far only a few penetration enhancers have been found to be effective in facilitating mucosal administration of large molecular drugs and have reached the market. Reasons for this include lack of a satisfactory safety profile respecting irritation, lowering of the barrier function, and impairment of the mucocilliary clearance protective mechanism. Furthermore, most of the popular penetration enhancers impart an extremely bitter and unpleasant taste, which make them unsuitable for human consumption.

One of the most effective routes to increase the bioavailabilty of orally-administered insulin, either by enhancing the absorption through the mucosa, or imparting a proper protection against the enzymatic degradation or both, is the use of liposomes and/or micelles as drug carriers. In this manner an improved absorption and thus a higher bioavailability can be obtained.

The conventional existing methods being used for the preparation of liposomes, however, suffer from one or more drawbacks. Most of them use pharmaceutically unacceptable toxic solvents, resulting in undesirable solvent residues, which cannot be acceptable for toxicological and environmental reasons. Despite their efficiency to form liposomes, a large number of these techniques have been developed on a laboratory scale and experience scale-up problems. Moreover, they involve high energy processes and expensive equipment. Likewise, the percentage entrapment achievable by some of the methods is also inherently very low.

According to a preferred embodiment of the present invention there is provided a novel method and formulation for spontaneous arrangement of any lipid-based structures such as liposomes (vesicles), micelles, lamellar structures (single or multilamellar), emulsions and any other liquid crystalline structures. These methods and formulations are intended for buccal delivery of insulin. By exposing the formulation according to the present invention to the saliva or any other liquid existing in the oral cavity, a spontaneous formation of liposomes and or micelles or any other possible structural arrangements of lipids, occurs. As a result, during the course of lipid arrangement, an in-situ insulin entrapment into the liposomes and/or micelles is obtained.

Although vesicles often form spontaneously in vivo, they have rarely been observed to form in vitro without the input of considerable mechanical energy (such as sonication or pressure filtration) or elaborate chemical treatments (detergent dialysis or reverse-phase evaporation). One of the earliest works regarding the concept of spontaneous formation of liposomes, is the study of Hauser et al (Proc. Int. Sch. Phys., “Enrico Fermi’, 90, (Phys. Amphiphiles), 648-662, 1985). They suggested a method based on the rapid, transient exposure of smectic phases of charged lipids to high pH (pH=11-12). After neutralization a stable lipid dispersion is obtained consisting of a mixture of LUV and SUV. The need of the lipids to be exposed to high pHs, however, prevents such systems from being used as a proper system for spontaneous liposome formation at physiological pHs. Karel and coworkers (Science, 245, 1371-1373, 1989) have suggested a new method for spontaneous vesicle formation. In this study spontaneous, single-walled, equilibrium vesicles were prepared from aqueous mixtures of simple, single-tailed cationic and anionic surfactant. There have been also other reports on spontaneous vesicle formation in certain mixtures of short and long-chain, double-tailed lecithins (Biochemistry, 23, 4011, 1984); in solutions of double-tailed surfactants with hydroxide and other more exotic counterions (Science, 221, 1047, 1983, J. Am. Chem. Soc., 106, 4279, 1984); in some mixtures of single-tailed surfactants (Biochemistry, 17, 3759, 1978); and in a mixture of egg yolk lecithin and cationic detergent in CHCl3/CH3OH solution (J. Am. Chem. Soc., 110, 971-973, 1988). Although these systems were an improvement over conventional sonicated vesicles, the relatively restricted chemical or physical properties of the vesicles or the limited availability of the surfactants were such that these methods were not widely exploited. Furthermore, most of these systems may be irrelevant for liposomes to be used as drug carriers, because of their detergent-like nature and, consequently, potential toxicity. The recent development of a spontaneous liposome forming-system, which is also marketed by Lucas Mayer under the trade name of “Pro-Liposome”, has been carried out by Wilks and his associates (European Patent 0158441). The pro-liposome mixture normally consists of a mixture of phospholipids dispersed in a hydrophilic medium which is aqueous ethanol. Formation of liposomes is enabled by addition of excess water. The loading of active ingredients is carried out by the addition of a low amount solution of the active ingredient into the proliposome mixture followed by a further addition of water enabling the formation of the liposomes. It was reported that by this manner generally oligo-or multilamellar vesicles with a void volume of at least 2 ml per gram of lipid, and capable of achieving a drug entrapment ratio of more than 20% can be obtained (European Patent 0158441).

With this state of the art in mind, there is now provided according to the present invention a solid composition for intra-oral delivery of insulin, comprising insulin; a hydrophilic polymer matrix; and a phospholipid providing insulin bioavailability of at least 5%.

In preferred embodiments of the present invention there is provided a solid composition for intra-oral delivery of insulin, comprising insulin; a hydrophilic polymer matrix; and a phospholipid, providing insulin bioavailability of at least 10%.

In especially preferred embodiments of the present invention there is provided a solid composition for intra-oral delivery of insulin, comprising insulin; a hydrophilic polymer matrix; and a phospholipid, providing insulin bioavailability of at least 15%.

In the most preferred embodiments of the present invention there is provided a solid composition for intra-oral delivery of insulin, comprising insulin; a hydrophilic polymer matrix; and a phospholipid, providing insulin bioavailability of at least 20%. In another aspect of the present invention there is provided a solid composition for intra-oral delivery of insulin, comprising insulin; a hydrophilic polymer matrix; and a liposome forming agent, wherein the composition achieves a bioavailability of insulin of at least 5%.

In preferred embodiments of this aspect of the present invention there is provided a solid composition for intra-oral delivery of insulin, comprising insulin; a hydrophilic polymer matrix; and a liposome forming agent, wherein the composition achieves a bioavailability of insulin of at least 10%.

In especially preferred embodiments of the present invention, there is provided a solid composition for intra-oral administration of insulin, comprising Insulin, a hydrophilic polymer matrix, and a phospholipid; wherein upon contact with the oral cavity liquid, said composition forms in-situ particles selected from the group consisting of micelles, emulsions, liposomes, or mixed structures thereof.

Thus the present invention provides a solid composition for intra-oral delivery of insulin, comprising insulin, a hydrophilic polymer matrix and a phospholipid; wherein upon contact with the oral cavity liquid, said composition forms in-situ particles that enhance the absorption of insulin selected from the group consisting of: micelles, emulsions, liposomes and/or mixed structures thereof.

Preferably the solid compositions according to the present invention are adapted for absorption of insulin via buccal mucosa, lingual mucosa and/or sublingual mucosa.

Thus the present invention preferably provides a solid composition as defined adapted for intra-oral absorption of insulin via buccal mucosa, lingual mucosa and/or sublingual mucosa.

According to preferred embodiments, the formulation comprises at least one hydrophilic polymer. According to specific embodiments of the present invention, the hydrophilic polymer is water-soluble polymer which is selected from the group consisting of a Povidone (PVP: polyvinyl pyrrolidone), polyvinyl alcohol, copolymer of PVP and polyvinyl acetate, HPC (hydroxypropyl cellulose), HPMC (hydroxypropyl methylcellulose), carboxymethyl cellulose, hydroxyethyl cellulose, hydroxylmethyl cellulose, methylcellulose, gelatin, proteins, collagen, hydrolyzed gelatin, polyethylene oxide, acacia, dextrin, magnesium aluminum silicate, starch, a water soluble synthetic polymer, polyacrylic acid, polyhydroxyethylmethacrylate (PHEMA), polyacrylamid, polymethacrylates and their copolymers, gum, water soluble gum, polysaccharide, hydroxypropylmethyl cellulose phthalate, polyvinyl acetate phthalate, cellulose acetate phthalate, hydroxypropylmethyl cellulose acetate succinate, poly(methacrylic acid, methyl methacrylate)1:1 and poly(methacrylic acid, ethyl acrylate)1:1, alginic acid, and sodium alginate, and any other pharmaceutically acceptable polymer that dissolves in buffer phosphate pH >5.5 and/or mixtures thereof.

In certain embodiments, gums include, for example and without limitation, heteropolysaccharides such as xanthan gum(s), homopolysaccharides such as locust bean gum, galactans, mannans, vegetable gums such as alginates, gum karaya, pectin, agar, tragacanth, accacia, carrageenan, tragacanth, chitosan, agar, alginic acid, other polysaccharide gums (e.g. hydrocolloids), and mixtures of any of the foregoing. Further examples of specific gums which may be useful in the formulation according to the present invention include but are not limited to acacia catechu, salai guggal, indian bodellum, copaiba gum, asafetida, cambi gum, Enterolobium cyclocarpum, mastic gum, benzoin gum, sandarac, gambier gum, butea frondosa (Flame of Forest Gum), myrrh, konjak mannan, guar gum, welan gum, gellan gum, tara gum, locust bean gum, carageenan gum, glucomannan, galactan gum, sodium alginate, tragacanth, chitosan, xanthan gum, deacetylated xanthan gum, pectin, sodium polypectate, gluten, karaya gum, tamarind gum, ghatti gum, Accaroid/Yacca/Red gum, dammar gum, juniper gum, ester gum, ipil-ipil seed gum, gum talha (acacia seyal), and cultured plant cell gums including those of the plants of the genera: acacia, actinidia, aptenia, carbobrotus, chickorium, cucumis, glycine, hibiscus, hordeum, letuca, lycopersicon, malus, medicago, mesembryanthemum, oryza, panicum, phalaris, phleum, poliathus, polycarbophil, sida, solanum, trifolium, trigonella, Afzelia africana seed gum, Treculia africana gum, detarium gum, cassia gum, carob gum, Prosopis africana gum, Colocassia esulenta gum, Hakea gibbosa gum, khaya gum, scleroglucan, zea, mixtures of any of the foregoing, and the like

In other embodiments according to the present invention the hydrophilic polymer may be water insoluble but water swellable polymer. The swellable polymer may be more preferably selected from the groups consisting of a water insoluble cross-linked polysaccharide, a water insoluble polysaccharide, a water insoluble synthetic polymer, a water insoluble cross-linked protein, a water insoluble cross-linked peptide, water insoluble cross-linked gelatin, water insoluble cross-linked hydrolyzed gelatin, water insoluble cross-linked collagen, water insoluble cross linked polyacrylic acid, water insoluble cross-linked cellulose derivatives, water insoluble cross-linked polyvinyl pyrrolidone, micro crystalline cellulose, insoluble starch, micro crystalline starch and a combination thereof. The water insoluble cross-linked polysaccharide is preferably, selected from the group consisting of insoluble metal salts or cross-linked derivatives of alginate, pectin, xantham gum, guar gum, tragacanth gum, locust bean gum, carrageenan, and metal salts thereof, and covalently cross-linked derivatives thereof. The modified cellulose is preferably, selected from the group consisting of cross-linked derivatives of hydroxypropylcellulose, hydroxypropylmethylcellulose, hydroxyethylcellulose, methylcellulose, hydroxymethyl cellulose, carboxymethylcellulose, and metal salts of carboxymethylcellulose.

In another embodiment according to the present invention the hydrophilic polymer may be a polymeric blend consisting of a combination of at list a water soluble polymer and at least a water insoluble but swellable polymer.

According to preferred embodiments, the formulation comprises at least one liposome forming agent. The liposome forming agent is selected from the group consisting of egg phosphatidylcholine (PC), dilauryl phosphatidylcholine (DLPC), dimyristoyl phosphatidylcholine (DMPC), dipalmitoyl phosphatidylcholine (DPPC), dioleoyl phosphatidylcholine (DOPC), dimyristoyl phosphatidylglycerol (DMPG), dipalmitoyl; phosphatidylglycerol(DPPG), dimyristoyl phosphatidic acid(DMPA), dipalmitoyl phosphatidic acid (DPPA), dipalmitoyl phosphatidylethanolamine (DPPE), distearoyl phosphatidylcholine (DSPC), brain phosphatidylserine (PS), brain sphingomyelin (SM), cholesterol(C), cardiolipin (CL), trioctanoin (TC), triolein (TO), soy phosphatidylcholine, poly(adenylic acid), phosphatidylethanolamine (PE), phosphatidyl glycerol (PG), phosphatidyl inositol (PI), sphingosine, cerebroside (glycolipid), and/or the combinations thereof.

In another embodiment the formulation contains at least one absorption enhancer, especially absorption enhancers selected from the group consisting of Na-salicylate-chenodeoxy cholate, Na deoxycholate, polyoxyethylene 9-lauryl ether, chenodeoxy cholate-deoxycholate and polyoxyethylene 9-lauryl ether, monoolein, Natauro-24,25-dihydrofusidate,Na-taurodeoxycholate,Na-glycochenodeoxycholate, oleic acid, linoleic acid, linolenic acid, polyoxyethylene ethers, polyoxyethylene sorbitan esters, polyoxyethylene 10-lauryl ether, polyoxyethylene 16-lauryl ether, azone(1-dodecylazacycloheptane-2-one), and sodium chloride, sodium bicarbonate in combination with the above mentioned materials.

According to preferred embodiments of the present invention, In order to prevent the degradation and oxidation of the active material the formulation may further comprise an antioxidant. Preferably, the antioxidant is selected from the group consisting of 4,4 (?,3 dimethyl tetramethylene dipyrochatechol), Tocopherol-rich extract (natural vitamin E), α-tocopherol (synthetic Vitamin E), β-tocopherol, γ-tocopherol, δ-tocopherol, Butylhydroxinon, Butyl hydroxyanisole (BHA), Butyl hydroxytoluene (BHT), Propyl Gallate, Octyl gallate, Dodecyl Gallate, Tertiary butylhydroquinone (TBHQ), Fumaric acid, Malic acid, Ascorbic acid (Vitamin C), Sodium ascorbate, Calcium ascorbate, Potassium ascorbate, Ascorbyl palmitate, Ascorbyl stearate, Citric acid, Sodium lactate, Potassium lactate, Calcium lactate, Magnesium lactate, Anoxomer, Erythorbic acid, Sodium erythorbate, Erythorbin acid, Sodium erythorbin, Ethoxyquin, Glycine, Gum guaiac, Sodium citrates (monosodium citrate, disodium citrate, trisodium citrate), Potassium citrates (monopotassium citrate, tripotassium citrate), Lecithin, Polyphosphate, Tartaric acid, Sodium tartrates (monosodium tartrate, disodium tartrate), Potassium tartrates (monopotassium tartrate, dipotassium tartrate), Sodium potassium tartrate, Phosphoric acid, Sodium phosphates (monosodium phosphate, disodium phosphate, trisodium phosphate), Potassium phosphates (monopotassium phosphate, dipotassium phosphate, tripotassium phosphate), Calcium disodium ethylene diamine tetra-acetate (Calcium disodium EDTA), Lactic acid, Trihydroxy butyrophenone, Deteroxime mesylate, and Thiodipropionic acid.

The formulation may further include a chelating agent to increase chelation of trace quantities of metals thereby helping in preventing the loss of the active material by oxidation. Preferably, the chelating agent is selected from the group consisting of Antioxidants, Dipotassium edentate, Disodium edentate, Edetate calcium disodium, Edetic acid, Fumaric acid, Malic acid, Maltol, Sodium edentate, Trisodium edetateMost preferably, the chelating agent is citric acid.

According to some embodiments of the present invention, the formulation may further comprise a synergistic agent (sequestrate). Preferably, the sequestrate is selected from the group consisting of citric acid and ascorbic acid.

Without wishing to be limited by a single hypothesis, chelating agents and sequestrates may optionally be differentiated as follows. A chelating agent, such as (preferably) citric acid is intended to help in chelation of trace quantities of metals thereby assisting to prevent the loss of the active ingredient(s), by oxidation. A sequestrate such as (preferably) ascorbic acid, optionally and preferably has several hydroxyl and/or carboxylic acid groups, which can provide a supply of hydrogen for regeneration of the inactivated antioxidant free radical. A sequestrate therefore preferably acts as a supplier of hydrogen for rejuvenation of the primary antioxidant.

In another embodiment, an antifungal, antimicrobial agent selected from the group consisting of ethyl paraben, methyl paraben, propyl paraben, metacrezole and combinations thereof may also be added to the composition.

In addition to the foregoing, the formualtion may also include additional excipients such as lubricants, bioadhesive agents, plasticizers, antisticking agents, natural and synthetic flavorings and natural and synthetic colorants.

In preferred embodiments the formulation according to the present invention further contains at least one of a wetting agent, suspending agent, surfactant, and dispersing agent, or a combination thereof.

Examples of suitable wetting agents include, but are not limited to, poloxamer, polyoxyethylene ethers, polyoxyethylene sorbitan fatty acid esters (polysorbates), polyoxymethylene stearate, sodium lauryl sulfate, sorbitan fatty acid esters, benzalkonium chloride, polyethoxylated castor oil, docusate sodium.

Examples of suitable suspending agents include but are not limited to, alginic acid, bentonite, carbomer, carboxymethylcellulose, carboxymethylcellulose calcium, hydroxyethylcellulose, hydroxypropyl cellulose, microcrystalline cellulose, colloidal silicon dioxide, dextrin, gelatin, guar gum, xanthan gum, kaolin, magnesium aluminum silicate, maltitol, medium chain triglycerides, methylcellulose, polyoxyethylene sorbitan fatty acid esters (polysorbates), polyvinyl pyrrolidone (PVP), propylene glycol alginate, sodium alginate, sorbitan fatty acid esters, and tragacanth.

Examples of suitable surfactants include but are not limited to, anionic surfactants such as docusate sodium and sodium lauryl sulfate; cationic, such as cetrimide; nonionic, such as polyoxyethylene sorbitan fatty acid esters (polysorbates) and sorbitan fatty acid esters.

Examples of suitable dispersing agents include but are not limited to, poloxamer, polyoxyethylene sorbitan fatty acid esters (polysorbates) and sorbitan fatty acid esters.

The content of the wetting agent, surfactant, dispersing agent and suspending agent may optionally be in an amount of from about 0 to about 30% of the weight of the dry film of the formulation.

The formulation according to the present invention may also optionally feature a buffering agent, which is preferably selected from the group consisting of an inorganic salt compound and an organic alkaline salt compound. More preferably, the buffering agent is selected from the group consisting of potassium bicarbonate, potassium citrate, potassium hydroxide, sodium bicarbonate, sodium citrate, sodium hydroxide, calcium carbonate, dibasic sodium phosphate, monosodium glutamate, tribasic calcium phosphate, monoethanolamine, diethanolamine, triethanolamine, citric acid monohydrate, lactic acid, propionic acid, tartaric acid, fumaric acid, malic acid, and monobasic sodium phosphate.

In another aspect of the present invention there is provided a solid composition for intra-oral delivery comprising a pharmaceutically acceptable active agent; a hydrophilic polymer matrix; and a phospholipid, wherein the composition provides bioavailability of said pharmaceutically acceptable active agent of at least about 5% and said pharmaceutically acceptable active agent has a dissolution rate higher than that of the said hydrophilic polymer.

Also provided according to the present invention is a solid composition for intra-oral delivery of insulin comprising insulin, a hydrophilic polymer matrix and a phospholipid providing a reduction of blood glucose levels of a subject by at least 5%.

The invention also provides a solid composition comprising a hydrophilic polymer matrix, at least one phospholipid and insulin.

In preferred embodiments of the present invention there is provided a solid composition comprising a hydrophilic polymer matrix, lecithin and insulin providing the reduction of glucose blood level of a subject by at least about 5%.

The present invention also provides a solid composition comprising a hydrophilic polymer matrix, phosphotidylcholine and insulin providing the reduction of glucose blood level of a subject by at least about 5%.

Also provided according to the present invention is a solid composition as defined herein that provides a reduction of blood glucose levels of a subject by at least about 5%.

In another aspect of the present invention there is provided a method for the reduction of the blood glucose plasma levels of a subject by at least 5% comprising administering to said subject a solid composition comprising: insulin, a hydrophilic polymer matrix and a phospholipid.

The present invention also provides a method for treating Type I diabetes comprising the intra-oral use of a solid composition comprising: insulin, a hydrophilic polymer matrix and a phospholipid.

Also provided according to the present invention is a method for decreasing the need for at least one subcutaneous injection a day for Type I diabetes patients comprising the intra-oral use of a solid composition comprising: insulin, a hydrophilic polymer matrix and a phospholipid.

In preferred embodiments of the present invention there is provided a method for treating Type II diabetes comprising the intra-oral use of a solid composition comprising: insulin, a hydrophilic polymer matrix and a phospholipid.

Thus the present invention also provides a method for decreasing the need for at least one subcutaneous injection a day for Type II diabetes patients comprising the intra-oral use of a solid composition comprising: insulin, a hydrophilic polymer matrix and a phospholipid.

In an especially preferred embodiment of the present invention there is provided a drug delivery system comprising a solid composition, said composition comprising a hydrophilic, blended, single phase polymeric material having insulin and a phospholipid incorporated therein for oral transmucosal delivery of said insulin via intra-oral mucosa.

In said preferred embodiments said phospholipid is preferably selected from the group consisting of lecithin or phosphotidyl-cholin.

Preferably and optionally said material is a bioadhesive film.

Thus in preferred embodiments of the present invention, there is provided a drug delivery system comprising a hydrophilic bioadhesive blended single phase polymeric material having insulin and lecithin or phosphatidyl-cholin incorporated therein for oral transmucosal delivery of said insulin via intra-oral mucosa, wherein upon contact with saliva, said system forms in situ particles selected from the group consisting of micelles, emulsions and liposomes, incorporating said insulin, for enhancing the absorption thereof.

Preferably there is provided a drug delivery system as defined, adapted for oral transmucosal delivery via mucosa selected from the group consisting of buccal mucosa, lingual mucosa, and sublingual mucosa any other places relating to oral cavity.

In preferred embodiments of the present invention, the drug delivery system provides an oral viability of at least 5%.

Preferably there is provided according to the present invention, a drug delivery system comprising a hydrophilic bioadhesive blended single phase polymeric material having insulin and phospholipids incorporated therein for oral transmucosal delivery of said insulin via intra-oral mucosa wherein upon contact with saliva, said system forms in situ particles selected from the group consisting of micelles, emulsions and liposomes, incorporating said insulin, for enhancing the absorption thereof.

As stated, in its preferred embodiments, the present invention suggests a novel system for intra-oral (oral cavity) delivery of insulin utilizing a spontaneous formation of liposomes by the components constituting the system. The system is based on the unique combination of a hydrophilic water soluble polymer and a proper lipid. The principle of the system according to the present invention is based on the fact that exposure of the hydrophobic moieties of amphiphils to water or aqueous solutions is thermodynamically unfavorable. Protection of these portions from aqueous solutions is possible through self-aggregation of the amphiphils where the hydrophobic moieties have minimal contact with water molecules. Therefore, on the contact with aqueous media, above a certain critical concentration and above the gel to liquid crystalline phase transition temperature (Tc), phospholipids spontaneously self-aggregate to form globular structures i.e. liposomes and/or micelles. The present invention exploits the unique combination of a carefully chosen lipid and a water soluble polymeric matrix. Spontaneous formation of liposomes and/or micelles is activated by the simple wetting of the mixture where the polymeric matrix starts to be dissolved and consequently the lipid components of the mixture are arranged in the form of bilayers, which eventually enclose to the vesicle structure. Additionally such a system may result in spontaneous formation of micelles, and/or emulsions. This unique mixture can pre-include insulin which is supposed to partially or completely undergo entrapment into the spontaneously formed liposomes and/or micelles. This system has a number of important advantages over existing methods for preparation of liposomes and/or micelles being used for pharmaceutical applications. The main advantage of the system is the avoidance of use of unacceptable solvents that could give rise to undesirable toxic solvent residues. Likewise, the organic solvents may, in most cases, result in biologically-deactivation of insulin when the active material should pre-entrapped in the lipid film. Likewise, organic solvents may, in most cases, result in the biological deactivation of the insulin when the active material is pre-entrapped in the lipid film.

Additionally the system, according to the present invention, provides a proper solution to the problem of the hydration process of lipids, which is one of the major obstacles in scaling-up for many existing conventional methods of liposome and/or micelle preparation. This unique method is simple and is suitable for scaling-up for production purposes, since it does not require any energy-expensive steps such as evaporation, sonication, freeze drying etc., or other complicated apparatus which can induce limitations to the scale up process. The system according to the present invention, can be prepared as a polymeric sheet (film). Thus it will be stable and readily transportable, as well as being suitable for extended storage for subsequent in-situ liposome and/or micelle formation. Likewise, in contrast to other conventional methods in which the loading of liposomes and/or micelles with insulin is often difficult and in some cases impossible, the liposomes and/or micelles formed spontaneously according to the present invention can readily be loaded in situ with insulin. The loading of liposomes and/or micelles with insulin is out simply, in-situ, during the hydration process of the film, which can take place in situ by saliva or liquids existing in the buccal or oral cavity. Since the liposome formation takes place in-situ, this system also suggests a good solution to the physical stability problem that is a serious problem for almost all conventionally prepared liposomes and/or micelles. The possibility of spontaneous formation of liposomes and/or micelles from the system according to the present invention, and in-situ loading with insulin, imparts an attractive feature, which can be a unique advantage in using this system as a proper dosage form specially for buccal delivery of insulin. Several delivery systems were designed for buccal delivery of insulin where some of them comprise a combination of polymer and lipids. Following are description of some important ones.

U.S. Pat. No. 6,290,987 B1 [Generex] discloses a mixed liposome formulation comprising insulin, water, an alkali metal alkyl sulfate, at least one membrane mimetic, and at least one phospholipids. The formulation is applied using an aerosol delivery system for buccal delivery. The patent does not teach, however, any use of a solid polymeric composition for the deliver of insulin nor does it teach the use of self-formation liposomes occurring in situ in the oral cavity. Furthermore, the patent does not teach or suggest a bioadhesive, blended, single-phase polymeric material having insulin incorporated therein. Also, the patent does not teach a delivery system which can be responsible for retaining the liposomes in the oral cavity, or preventing swallowing of the liposome into the GI tract where the fluids can be significantly destructive to insulin.

U.S. Pat. No. 6,432,383 B1 [Generex] discloses a mixed micellar formulation which includes a micellar proteinic agent, an alkali metal lauryl sulfate, an alkali metal salicylate, an edentate, and at least one absorption enhancing compound. The invention is intended for buccal delivery of insulin. The invention does not, however, disclose a solid polymeric composition for the deliver of insulin nor the delivery system for self-formation micelles or the system providing retention of said micelles in the oral cavity.

U.S. Pat. No. 6,264,981 (WO 0130288) [Anesta]—Relates to a drug formulation comprising a solid pharmaceutical agent in solid solution with a dissolution agent. The formulation is administered into a patient's oral cavity, delivering the pharmaceutical agent by absorption through a patient's oral mucosal tissue. The formulation and method provide for improved oral mucosal delivery of the pharmaceutical agent. This invention also relates to the use of oral transmucosal patch. Insulin specifically as a possible pharmaceutical agent of the formulation has been mentioned. Claim 1 reads as follows: “An improved oral transmucosal solid dosage form drug delivery formulation comprising: a pharmaceutical agent capable of being absorbed into oral mucosal tissue having a dissolution rate in the solvents found in the oral cavity, a dissolution agent having a dissolution rate in the solvents found in the oral cavity, said dissolution rate of said dissolution agent being greater than said dissolution rate of said pharmaceutical agent, and said pharmaceutical agent being in solid solution with said dissolution agent.”

The invention relates to dissolution improvement of the drug molecules which is intended for delivery into the oral cavity where there is relatively little solvent into which a solid dosage form can dissolved. The invention is limited to solid solutions and does not relate to buccal delivery of insulin and also the self-formation of liposomes in situ in the oral cavity. The invention does not provide bioavailability of at least 5% of insulin.

WO 00/33817 [PHARES PHARMACEUTICAL RESEARCH N.V]—relates to a carrier for hydrophilic and particularly for hydrophobic compounds that has pharmaceutical and industrial applications. It provides compositions in non-liquid form that are easy to prepare, and that may be solid compacts or may be particulate. At least one solid hydrophilic substance, most preferably a polymer, is typically, included in the composition. At least one biologically active compound may be present in the lipid polymer associate. The lipid polymer associates have the potential to swell in water or other aqueous media to form viscous intermediate compositions, Hydration may take place in situ e.g. from powders or granules inside a hard capsule or from a tablet in the GI tract and other mucosal surfaces. Said application also does not teach or suggest a bioadhesive, blended, single-phase polymeric material having insulin incorporated therein. Similarly this application does not suggest a delivery device specifically for insulin delivery, in the oral cavity for modified buccal absorption.

WO 2004/080438 [CAMURUS AB]—relates to an orally administrable composition comprising at least one physiologically tolerable polymer having, dispersed therein, particles comprising at least one physiologically tolerable lipid and a bioactive agent (that may be hydrophilic), which particles on contact with water or GI tract liquid form nanometer-sized particles containing said lipid, said bioactive agent and water. The suggested composition according to this invention reveals a phase segregation in the solid phase which can result in a non-homogeneous mixture and thus not capable of forming a homogeneous film and does not teach or suggest a bioadhesive, blended, single-phase polymeric material having insulin incorporated therein. Likewise, the invention does not disclose a system for buccal delivery of insulin or any ratio of bloavailability of insulin.

WO 2004/041118 [UMD, INC.]—discloses a method for topical or systemic delivery of drugs to or through nasal, buccal, vaginal, labial or scrotal epithelium. Said method comprises a step of contacting the vaginal, nasal, buccal, labial or scrotal epithelium with a foam or film composition consisting essentially of a substrate polymer and a pharmacologically effective agent. The invention does not teach, the self-formation of liposomes for buccal delivery and absorption of insulin or any ratio of bioavailability of insulin.

While the invention will now be described in connection with certain preferred embodiments in the following examples and with reference to the accompanying figures so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus, the following examples which include preferred embodiments will serve to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as of the principles and conceptual aspects of the invention.

### IN THE DRAWINGS

FIG. 1 is the calibration curve of gel permeation chromatography analysis.

FIG. 2 is the typical electron micrographs (TEM) of negatively stained spontaneously formed liposomes from the wefting of ILFPM.

FIG. 3 is the typical electron micrographs (TEM) of negatively stained vesicles prepared using the conventional “thin lipid film” method.

FIG. 4 is the histograms of the size distribution of the liposomes formed from HPC/PC (weight ratio of 7:3).

FIG. 5 is the histograms of the size distribution of the liposomes formed from HPC/PC+cholesterol (weight ratio of 7:3)

FIGS. 6-10 show the results of confocal microscopy analysis of the dissolution and destruction of HPC in the process of spontaneous vesicle formation from the ILFPM.

FIGS. 11-24 show the results of confocal microscopy analysis of the spontaneous vesicle formation from the ILFPM via transformation of phospholipid to tubular fibril, penetration of water between the bilayers, vesiculation and dispersion of spontaneously formed liposomes processes.

FIGS. 25A and 25B are graphical representations of the effect of PC content in ILFPM on entrapment and entrapment efficiency.

FIG. 26 shows the effect of active material content on entrapment and entrapment efficiency.

FIGS. 27A and 27B show the effect of PC+CHL/HPC weight ratio in ILFPM on entrapment and entrapment efficiency.

FIG. 28 shows the effect of hydrating medium volume on entrapment and entrapment efficiency.

### EXAMPLES

### Example 1

### Preparation of an In-situ Liposome Forming Polymeric Matrix (ILFPM)

The ILFPMs were prepared using a solution casting method. Accordingly, Klucel (467 mg) was dissolved in ethanol (9 g) at room temperature using magnetic stirrer, at ˜500 rpm. Phospholipid (200 mg) was added to the HPC solution while stirring and the dissolution of the PL was accomplished at room temperature. Cholesterol (CHL), when needed in the formulation, was added to the Klucel solution after dissolving of Klucel and the temperature was raised to 50° C. until a complete dissolution of cholesterol was obtained. In this case the phospholipid was added to the formulation after cooling the solution to the room temperature. The active material was added to the solution after obtaining complete dissolution of all components of the formulation. The solution was then cast into a polyethylene weighing plate and ethanol was allowed to evaporate at room temperature for at least 48 hours. Table 1 summarizes the formulations which were prepared and assessed in the present study.

TABLE 1
The formulations which were used in the present study

| | PC + CHL/HPC | CHL/PC | AM/HPC | AM/PC + CHL | AM/PC + CHL | AM/For. | |
|---|---|---|---|---|---|---|---|
| Form. | weight ratio | molar ratio | weight ratio | weight ratio | % (w/w) | % (w/w) | AM |
| 285-108 | 3/7 | 0/100 | 4.1/95.9 | 9.1/90.9 | 10 | 2.9 | DHE |
| III/112 | | | | | | | |
| 285-108 | 3/7 | 0/100 | 4.1/95.9 | 9.1/90.9 | 10 | 2.9 | DHE |
| III/121 | | | | | | | |
| 285-125 | 100/0 | 0/100 | 100/0 | 9.1/90.9 | 10 | 9.1 | DHE |
| CTLFM | | | | | | | |
| 285-127 | 100/0 | 0/100 | 100/0 | 16.7/83.3 | 20 | 16.7 | Na-dic |
| CTLFM | | | | | | | |
| 285-128 | 100/0 | 0/100 | 100/0 | 9.1/90.9 | 10 | 9.1 | DHE |
| CTLFM | | | | | | | |
| 349-14/2 | 3/7 | 0/100 | 7.9/92.1 | 16.7/83.3 | 20 | 5.7 | flurbipro. |
| 349-29/2 | 26.2 + 3.8/70 | 22.2/77.8 | 7.9/92.1 | 16.7/72.7 + 10.6 | 20 | 5.7 | Na-dic |
| 349-29/4 | 26.2 + 3.8/70 | 22.2/77.8 | 7.9/92.1 | 16.7/72.7 + 10.6 | 20 | 5.7 | flurbipro. |
| 349-29/6 | 26.2 + 3.8/70 | 22.2/77.8 | 7.9/92.1 | 16.7/72.7 + 10.6 | 20 | 5.7 | Na-salic |
| 349-29/10 | 26.2 + 3.8/70 | 22.2/77.8 | 0/100 | 0/87.3 + 12.7 | 0 | 0 | — |
| 349-35/1 | 26.2 + 3.8/70 | 22.2/77.8 | 0/100 | 0/87.3 + 12.7 | 0 | 0 | — |
| 349-35/2 | 28.4 + 1.6/70 | 22.2/77.8 | 7.9/92.1 | 16.7/72.7 + 10.6 | 20 | 5.7 | Na-dic |
| 349-35/3 | 1/9 | 0/100 | 0/100 | 0/100 | 0 | 0 | — |
| 349-35/4 | 1/9 | 0/100 | 7.9/92.1 | 43.5/56.5 | 77 | 7.2 | Na-dic |
| 349-35/5 | 8.7 + 1.3/90 | 22.2/77 | 0/100 | 0/100 | 0 | 0 | — |
| 349-35/6 | 8.7 + 1.3/90 | 22.2/77.8 | 7.9/92.1 | 43.5/49.3 + 7.2 | 77 | 7.2 | Na-dic |
| 349-35/7 | 1/1 | 0/100 | 0/100 | 0/100 | 0 | 0 | — |
| 349-35/8 | 5/5 | 0/100 | 7.9/92.1 | 7.9/92.1 | 8.6 | 4.1 | Na-dic |
| 349-35/9 | 43.7 + 6.3/50 | 22.2/77.8 | 0/100 | 0/100 | 0 | 0 | — |
| 349-35/10 | 43.7 + 6.3/50 | 22.2/77.8 | 7.9/92.1 | 7.9/80.4 + 11.7 | 8.6 | 4.1 | Na-dic |
| 349-42/2 | 3/7 | 0/100 | 7.9/92.1 | 16.7/83.3 | 20 | 5.7 | Na-dic |
| 349-44/1 | 26.2 + 3.8/70 | 22.2/77.8 | 24.8/75.2 | 43.5/49.3 + 7.2 | 7.7 | 18.7 | Na-dic |
| 349-44/2 | 26.2 + 3.8/70 | 22.2/77.8 | 3.6/96.4 | 7.9/80.4 + 11.7 | 8.6 | 2.5 | Na-dic |
| 349-47/3 | 3/7 | 0/100 | 7.9/92.1 | 16.7/83.3 | 20 | 5.7 | sulindac |
| 349-47/4 | 26.2 + 3.8/70 | 22.2/77.8 | 7.9/92.1 | 16.7/72.7 + 10.6 | 20 | 5.7 | sulindac |
| 349-47/7 | 28.4 + 1.6/70 | 10/90 | 7.9/92.1 | 16.7/72.7 + 10.6 | 20 | 5.7 | Na-dic |
| 349-47/8 | 28.4 + 1.6/70 | 30/70 | 7.9/92.1 | 16.7/72.7 + 10.6 | 20 | 5.7 | Na-dic |
| 349-47/9 | 3.7 | 0/100 | 0/100 | 0/100 | 0 | 0 | — |
| 349-47/10 | 28.4 + 1.6/70 | 22.2/77.8 | 0/100 | 0/87.3 + 12.7 | 0 | 0 | — |
| 349-64/1 | 3/7 | 0/100 | 2.1/97.9 | 4.8/95.2 | 5 | 1.5 | flurbipro |
| 349-64/2 | 26.2 + 3.8/70 | 22.2/77.8 | 2.1/97.9 | 4.8/83.1 + 12.1 | 5 | 1.5 | flurbipro |
| 349-64/3 | 3/7 | 0/100 | 2.1/97.9 | 4.8/95.2 | 5 | 1.5 | sulindac |
| 349-64/4 | 26.2 + 3.8/70 | 22.2/77.8 | 2.1/97.9 | 4.8/83.1 + 12.1 | 5 | 1.5 | sulindac |
| 349-69 | 100/0 | 0/100 | 100/0 | 4.8/83.1 + 12.1 | 20 | 16.7 | flurbipro |
| 349-72/1 | 3/7 | 0/100 | 7.9/92.1 | 16.7/83.3 | 20 | 5.7 | Na-dic |
| 349-72/2 | 28.4 + 1.6/70 | 22.2/77.8 | 7.9/92.1 | 16.7/83.3 | 20 | 5.7 | Na-dic |
| 349-72/7 | 3/7 | 0/100 | 2.1/97.9 | 16.7/72.7 + 10.6 | 5 | 1.5 | Na-salic |
| 349-72/8 | 26.2 + 3.8/70 | 22.2/77.8 | 2.1/97.9 | 4.8/95.2 | 5 | 1.5 | Na-salic |
| 349-87/9 | 6/4 | 0/100 | 7.9/92.1 | 4.8/83.1 + 12.1 | 5.7 | 3.3 | Na-dic |
| 349-87/10 | 8/2 | 0/100 | 7.9/92.1 | 5.4/94.6 | 2.1 | 1.7 | Na-dic |
| 349-87/11 | 0/100 | 0/0 | 7.9/92.1 | 2.1/97.9 | 100 | 7.9 | Na-dic |
| 349-97/1 | 3/7 | 0/100 | 7.9/92.1 | 100/0 | 20 | 5.7 | Na-dic |
| 349-97/2 | 26.2 + 3.8/70 | 22.2/77.8 | 7.9/92.1 | 16.7/83.3 | 20 | 5.7 | Na-dic |
| 349-97/7* | ¹27 + 3/70 | 0/100 | 7.9/92.1 | 16.7/72.7 + 10.6 | ³20 | 5.7 | Na-dic |
| 349-97/9* | ¹27 + 3/70 | 0/100 | 7.9/92.1 | ²16.7/75 + 8.3 | ³20 | 5.7 | Na-salic |
| 349-97/11* | ¹27 + 3/70 | 0/100 | 7.9/92.1 | ²16.7/75 + 8.3 | ³20 | 5.7 | sulindac |
| 349-97/13* | ¹27 + 3/70 | 0/100 | 7.9/92.1 | ²16.7/75 + 8.3 | ³20 | 5.7 | flurbipro |
| 349-97/14* | ¹28.5 + 1.5/70 | 0/100 | 2.1/97.9 | ²16.7/75 + 8.3 | ³20 | 5.7 | flurbipro |
| 349-103/1* | ¹27 + 3/70 | 0/100 | 7.9/92.1 | ²16.7/79.2 + 4.1 | ³5 | 1.5 | flurbipro |
| 348-103/6 | 0/100 | 0/0 | 7.9/92.1 | ²4.8/85.7 + 0.5 | 100 | 7.9 | Na-salic. |
| 349-103/7 | 0/100 | 0/0 | 7.9/92.1 | 100/0 | 100 | 7.9 | sulindac |
| 349-103/8 | 0/100 | 0/0 | 7.9/92.1 | 100/0 | 100 | 7.9 | flurbipro. |
| 350-32 | 100/0 | 0/100 | 100/0 | 100/0 | 20 | 16.7 | Na-dic |
| AMTLFM | | | | | | | |
| 350-321 | 100/0 | 0/100 | 100/0 | 16.7/83.3 | 20 | 16.7 | sulindac |
| BMTLFM | | | | | | | |
| 350-327 | 100/0 | 0/100 | 100/0 | 16.7/83.3 | 20 | 16.7 | Na-salic. |
| CMTLFM | | | | | | | |

PC—Phosphatidylcholine, CHL—Cholesterol, AM—Active material, HPC—Hydroxypropyl cellulose; For.—Formulation, Na-salic.—Na-salicylate, Na-dic.—Na-diclofenac, flurbipro.—flurbiprofen, PS—Phosphatidylserine, CTLFM—Classic thin lipid film method, MTLFM—Modified thin lipid film method.
*Formulation containing PS with no CHL.
¹The weight ratio of PC + PS/HPC.
²The weight ratio of AM/PC + PS.
³Weight percent of AM/PC + PS/

### Example 2

### Preparation of In-situ Liposome Forming Polymeric Matrix (ILFPM) Containing Insulin

The Insulin-containing system was prepared using a solution casting method.

Insulin solution (3.0 g), containing 100u/ml insulin, m-cresol and glycerol was diluted with purified water (3.6 g). Sodium Lauril Sulphate (0.113 g) was dissolved in the solution, at room temperature using a magnetic stirrer, at about 500 rpm. Ethanol (4.8 g), was added. Klucel L (0.56 g) was dissolved in the solution at room using a magnetic stirrer, at about 500 rpm. Phospolipid (Epikuron 200, 0.24 g) was added to the solution while stirring at room temperature. The solution was then cast into a polyethylene weighing plate and the solvents were allowed to evaporate at room temperature for at last 48 hours. Table 2 summarizes insulin-containing formulations.

TABLE 2
Insulin-containing ILFPM formulation

| Components | mg/20 U | % |
|---|---|---|
| Insulin | 0.800 | 1.3% |
| HPC | 37.360 | 59.8% |
| Epicuron 200 | 16 | 25.6% |
| SLS | 7.52 | 12.0% |
| Flavor additives | 0.8 | 1.3% |
| Total | 62.480 | 100.0% |

Ratio HPC/PC 70/30
Ratio HPC/PC/Ins 69/29.5/1.5
Ratio Ethanol/water

### Example 3

### Transmission Electron Microscopy (TEM)

Samples for negative staining were prepared by wetting of a small piece of film, placed on a glass micro slide, by adding one drop of distilled water initiating the dissolution of the polymer and spontaneous formation of liposomes. After about 5 minutes when an opaque concentrated liposome suspension was obtained a drop of the suspension, from the region in the boundary between the suspension and water, was transferred to a thin carbon-coated collodion film supported on a grid. An aqueous solution of 2% ammonium molybdate was used for negative staining of the liposomes. A drop of this negative staining solution was placed on the sample for at least 10 minutes. The excess liquid was removed by adsorption onto a filter paper. All samples were examined in a CM 12 Philips.

### Example 4

### Confocal Microscope

### A. Preparation of PE-Fluorescein-Containing Samples

HPC (630 mg) was first dissolved in ethanol (7 g) using a magnetic stirrer (500 rpm) at 40° C.-60° C. For the CHL-containing formulations, the CHL was added (34.3 mg) to the solution at the same temperature. After complete dissolution of HPC (and CHL), the solution was allowed to cool to room temperature. PC (270 mg or 235.7 mg for the formulations without and with CHL respectively) was added while stirring to obtain a homogeneous and clear solution with a weight ratio of 7:3 of HPC to PC (or PC+CHL). PE-fluorescein (1 mg) was separately dissolved in ethanol (2 ml) by hand shaking, at room temperature and the solution was then kept at 4° C. in a vial covered with aluminum foil. Of the former solution 1700 μl and of the latter solution (PE-fluorescein solution) 172 μl or 150 μl, for the formulations without and with CHL respectively, were mixed together. The mixed solution was finally cast into a polyethylene weighing plate which was left in the dark oven at 18° C. until complete evaporation of ethanol was obtained.

### B. Preparation of the Samples for Confocal Microscopy Observations:

The confocal microscopy observations were performed using Confocal Laser Scanning Microscope, Zeiss 410. In order to prevent the bleaching of the samples during the observation a mounting solution which contained (w/w) 80% glycerol, 20% PBS (pH=9.0), 3% Dabco, and 0.1% sodium azide, was added (one drop) to the dried PE-fluorescein-containing samples placed on a glass micro slide a few seconds prior to the hydration of the films. The observations were performed on both dry and wet samples, where distilled water (3-5 drops) was used for wetting of the samples 5 minutes prior to the observation.

### Example 5

### Trapped Volume Determination

The trapped volume of the spontaneously formed vesicles was determined by preparing ILFPM containing 6-caboxyfluorescein (6-FAM). CHL (25.4 mg) was first dissolved in ethyl alcohol (9 g) at 40° C. and then HPC (467 MG) was added to the solution. After complete dissolution of HPC, PC (Epikuron 200, 174.6 mg) was added and completely dissolved in the solution. A solution of 6-FAM (1.3 ml, 31 ppm) in Tris buffer (pH=7.5) was added. In all cases the addition and dissolution of materials was carried out while stirring at room temperature. The solution was then cast into a polyethylene weighing plate and ethanol was allowed to undergo evaporation at room temperature for at least 48 hours. The hydration of the 6-FAM containing films was performed using 1 ml of Tris buffer (25 μM). The hydrated films were then placed at 37° C. for overnight (18 hours). The separation of spontaneously formed liposomes from the aqueous medium (supernatant) was carried out by centrifugation at 18000 rpm for 1.5 h at 20° C. using a Sorvall Super T 21 centrifuge. The residues of the supernatant solution were carefully removed with a swab. The absorbance intensity of the trapped solution was measured after addition (1 ml) of Triton X-100 (10%). The concentrations of both supernatant 6-FAM solution as well as trapped solution in precipitate were determined using a calibration curve prepared in the range of 0.0620-10.3300 ppm. The absorbance measurements were performed spectrophotometrically at 480 nm using HP 8452A Diode-Array. The volume of the total internal aqueous compartment (Vi) of the vesicle was calculated from the amount of trapped solute, the concentration of the trapped solute in the supernatant (C1), and the molar concentration of phospholipid (CMpc) using the following correlation (Roseman, A. M., Lentz, B. R., Sears, B., Gibbes, D., Thompson, T. E., Chem. Phys. Lipids, 21, 205-222,1978).
Vi=[C2*V2/(C1−C2)]/CMpc, where C2 is the concentration of trapped solute measured after addition of Triton, and V2 is the volume of Triton added to the precipitated liposomes.

### Example 6

### Entrapment Assessments

The percent entrapment and entrapment efficiency were examined for several active materials representing each of the groups of very water soluble, intermediate, and very low soluble active materials. The percent entrapment (A_{L}/A_{T}* 100%) is defined as the total amount of drug/agent associated with the liposomes (A_{L}), divided by the total amount of drug/agent used during the preparation of ILFPM (A_{T}). The entrapment efficiency is defined as the ratio between the concentration of encapsulated drug/agent and the concentration of lipid used in the ILFPM formulation. The active material normally was added into the solution of ILFPM formulation and the solution was cast into a polyethylene weighing plate to result in a dry film which finally included the active material. Either Tris buffer (0.5 μM) or phosphate buffer, intestinal fluid TS ((pH=7.4) (IF TS), was used for ILFPM hydration and dissolving of the HPC (suspension medium or hydrating medium). A predetermined volume of buffer was added to the film weighing approximately (but accurately) 40 mg. After complete dissolution, the suspension was centrifuged at 17500 rpm for 1 hour at 20° C. using a Sorvall Super T 21 whereby the liposomes precipitated while the free active material remained dissolved in the supernatant.

The hydration of ILFPM containing DHE was carried out using buffer citrate-HCl (pH=2) so that the concentration of total DHE was 232 ppm. 5 ml of acidic buffer were added to the film weighing approximately (but accurately) 40 mg. The films were incubated at room temperature for either overnight or for 1 hour followed by gentle agitation by hand for 5 minutes. The separation of encapsulated and free DHE was carried out by centrifugation as described above.

The concentration of active materials in both supernatant as well as the precipitate was determined using a HP 8452A Diode-Array Spectrophotometer at 260 nm, 328 nm, 248 nm, 280 nm, and 296 nm for sodium diclofenac, sulindac, flurbiprofen, DHE and sodium salicylate respectively. The calibration curves obtained from the standard solutions, in intestinal fluid TS in the concentration range of 0-50 ppm, 2-60 ppm, 1-20 ppm, 0-30 ppm, and 2-20 ppm were respectively used for determination of sodium diclofenac, sulindac, flurbiprofen, DHE, and sodium salicylate concentrations. To determine the amount of the active material found in the precipitate, first the precipitate was entirely dissolved in ethanol and then the concentration was determined in the ethanol solution. The entrapment of some active materials was also assessed where the loading process of the liposome with active material was carried out from active material solution (AM.Sol.) which was also used for wetting and dissolving of the ILFPM containing no active material. In this case the procedure of the wetting, dissolution, separation between encapsulated and non-encapsulated active material, and determination of the active material concentration was the same as described above. The effect of the volume of the buffer used for hydration and dissolution, was checked by using varying volumes of the buffer added onto the ILFPMs. In this case the rest of the procedures were the same as described above. The effect of the two-step addition of the buffer onto the film was assessed using the same procedures.

### Example 7

### Gel Exclusion (Ael Filtration) Chromatography

Gel filtration chromatography was used to separate encapsulated and free DHE from each other. The column was prepared using Sepharose CL-2B (Lot #Q1-12374) which was supplied by Pharmacia. According to this method the separation takes place according to the size of the components. Accordingly, the liposomes, because of their size, are the first fractions being excluded in the void volume of the column. The free drug is excluded in the subsequent volumes, i.e. column's volume. The benefit of this method is that the column's washing dilutes the loaded liposomal sample and increases the probability of complete dissolution of the unloaded DHE. The volume of the column was approximately 8 ml and the volume of the loaded liposomal sample was 0.2 ml. The washing medium was buffer citric acid-NaOH-HCl at pH2, which was degassed by helium prior to use. The separation was performed at room temperature. Fractions (20 fractions) with a volume of 1.45 ml were collected in each separation process.

### Example 8

### Gel Permeation Chromatography

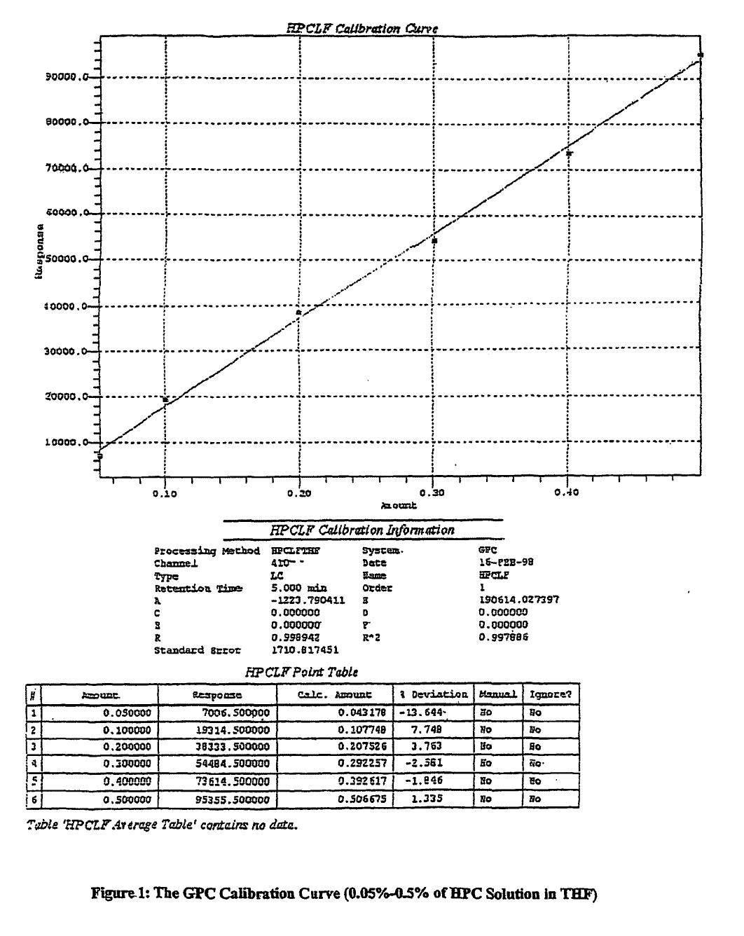

Gel permeation chromatography (GPC) method was used to assay the entrapment of HPC in the liposomes formed spontaneously. The HPC entrapment was determined by determining the amount of HPC in the precipitate obtained after centrifugation (at 17500 for 1.5 h, at room temperature) of the suspension resulted after hydration of ILFPM weighed accurately in the range of 60-90 mg. The hydration of the samples was performed using 5 ml intestinal fluid TS. at room temperature by hand shaking for about 15 minutes. The samples of the precipitate were prepared by dissolving the precipitate in THF (3 ml). The amount of HPC found in the precipitate, after the centrifugation process, was quantified using a calibration curve prepared in the range of 0.05%-0.5%. The GPC system consisted of a Waters 510 HPLC pump, a Waters 410 Differential Refractometer (at 40° C.), a Waters 717 Autosampler, and a Waters column heater (35° C.). A PL gel 5μ, 10 A column was used for GPC analysis. LiChrosolv THF was used as mobile phase which was carefully degassed (by helium gas and sonication for 2 minutes) prior to use and filtered on-line through a Rheodyne inlet filter before the column. Both standards as well as the samples solutions were filtered through a 0.45 μ syringe filter prior to injection. An injection volume of 30 μl was used in both cases of the samples as well as the standards. The mobile phase flow rate of 1 ml/min was kept throughout the analysis. The calibration curve is shown in FIG. 1.

### Example 9

### Size and Size Distribution Measurements

The average diameter and size distribution of the ILFPM-based vesicles were measured using a sub-micron particle analyzer, Coulter model N4MD, with a size distribution processor analysis and multiple scattering angle detection. Approximately, but accurately, 1.5 mg of ILFPM sample was first suspended in 0.5 ml distilled water which was allowed to form a homogeneous suspension after completely dissolving HPC by either gently hand shaking or short Vortex shaking for varying period of times at room temperature. A volume of 10-50 μl, depending on the counts/sec of the instrument, was taken from the suspension and diluted by 3 ml of distilled water. The analysis was carried out at 25° C. and dust (background) of 0% was obtained before the analysis. A viscosity of 0.849 CP and refractive index of 1.33 were considered throughout the analysis.

### Example 10

### Preparation of Liposomes Using “Modified Thin Lipid Film” Method (MTLFM)

The principle of modified thin lipid film method is based on formation of drug/lipid film, by drying down of a phospholipid solution, and hydration of resulted thin lipid film by hand shaking. The starting point was lipid solution preparation, which took place by the dissolving of phospholipid (100 mg of Epikuron 200) and the active material (20 mg) in ethanol (40 ml) in a 250 ml round-sided glass vessel. In order to increase the surface area available for formation of the thin lipid film (drug/lipid film) and thus to enable the hydration process to be carried out easily, glass beads (3.5 mm, 2 g) were added to the lipid/drug solution. The drying process of the solution was carried out in a rotary evaporator fitted with a cooling coil and a thermostatically controlled water bath. The evaporation of solvent was carried out at 50° C. under reduced pressure. The rotation velocity was 150-200 rpm. This procedure resulted finally in a thin lipid film dried onto both the sides of the glass vessel as well as the glass beads. The hydration of the thin lipid film was carried out by mechanical dispersion which is commonly known as the ‘hand-shaking’ method. For this purpose intestinal fluid TS (pH=7.5) (40 ml), which was preheated to 50° C., was added to the thin lipid film and the vessel was shaken by hand for 10-15 minutes until a homogeneous suspension was obtained. The entrapment of the drug was determined as described in the section of drug entrapment assessment.

### Example 11

### Preparation of Liposomes Using “Classic Thin Lipid Film” Method (CTLFM)

PC (phosphatidylcholine of soybean origin 95%, S-100, LOT #790129-1, supplied by Lipoid) and DHE or Na-diclofenac were dissolved in 45-100 ml ethanol in a round bottom flask of 1000 ml. The solution was dried by a rotary evaporator apparatus for 3 hours at room temperature to form a thin lipid film onto the sides of the flask. The hydration of the thin lipid film containing DHE was carried out using either buffer citrate-HCl (pH-2) or buffer citric acid NaOH-HCl (pH-2), so that the concentration of total DHE was 232 ppm. The separation of the liposomes and the unencapsulated drugs was carried out in the same way as described above for ILFPM method.

### Example 12

### Release Assessment of Active Material From ILFPM-Based Liposomes

Two ILFPM formulations (with and without CHL, 349-72/2, 349-72/1 respectively) containing Na-diclofenac as active material were used for this purpose. The ILFPM films were hydrated using 1 ml intestinal fluid TS (pH=7.5) at room temperature. The films were allowed to form the liposomes suspension, with no shaking, for various periods of time (0.25, 0.5, 3, and 24 hours). The Na-diclofenac concentration was spectrophotometrically determined as mentioned for the entrapments assessment. Duplicate films were used for each period of time. The concentration of released active material in the supernatant was determined after centrifugation of the suspensions as described for drug entrapment assessment.

### Example 13

### Characterization of Spontaneously Formed Liposomes

### 13.1 TEM Results

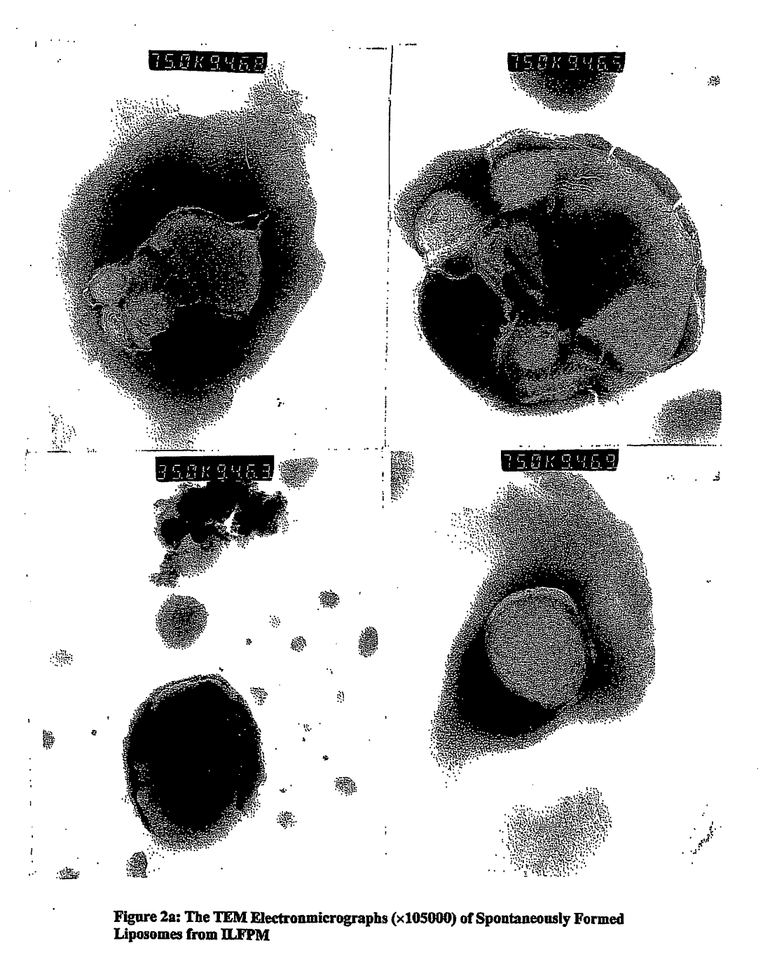

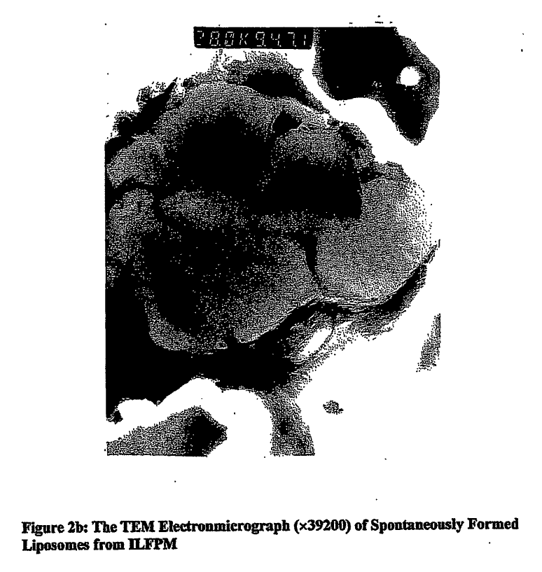

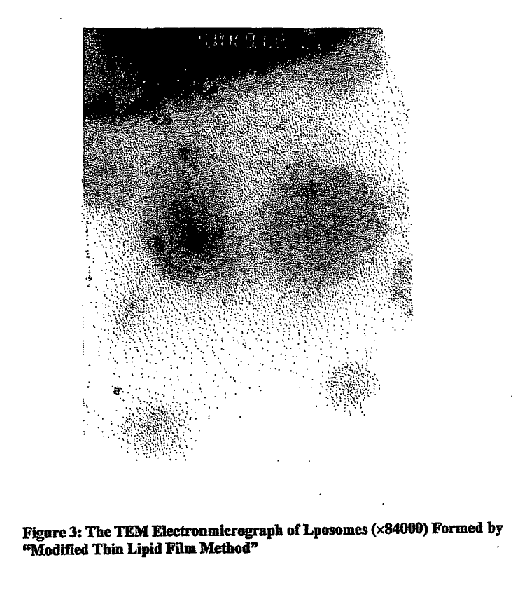

The typical electronmicrographs of negatively stained spontaneously formed liposomes from the wetting of ILFPM (formulations 349-47/9,10) are presented in FIG. 2. FIG. 2 indicates that the liposomes meshed spontaneously from ILFPM are normally oligo- or multilamellar. Multilamellar staining pattern is characteristic of phospholipids in the bilayer phase, suggesting that the membranes forming the walls consist of several phospholipid bilayers. It can be observed also that the walls of the vesicles usually appeared as broad poorly-defined bands, ranging in thickness from 160 to 450Å. This multilamellar structure can also be formed for the vesicles prepared using the conventional “thin lipid film” method, as it can be seen in FIG. 3. The fact that MLVs are the main product obtained spontaneously upon the hydration of ILFPM, can be naturally predictable since MLVs have slightly higher free energies than hydrated precipitate (phospholipid aggregate) and significantly lower than both LUVs as well as SUVs. Therefore, MLVs are formed normally first upon the exposure of uncharged phospholipid to water or any aqueous media and in order to achieve LUVs and SUVs more energy (swirling, shaking, vortexing, sonicating etc.) must be dissipated into the system. This fact has been described in more detail elsewhere (Lasic, D. D., Biochem., J., 256, 1-11, 1988).

The aggregates of liposomes observed in FIG. 2 can be the results of the spreading of monolayer liposomes embedded in negative stain across the grid. This is in fact the main problem of the negative staining electron microscopy method where the heavy metal stains lead to aggregation and possible re-organization of liposomes (“Liposomes, A Practical Approach”, The Practical Approach, R. R. C.).

### 13.2. Particle Size Analysis:

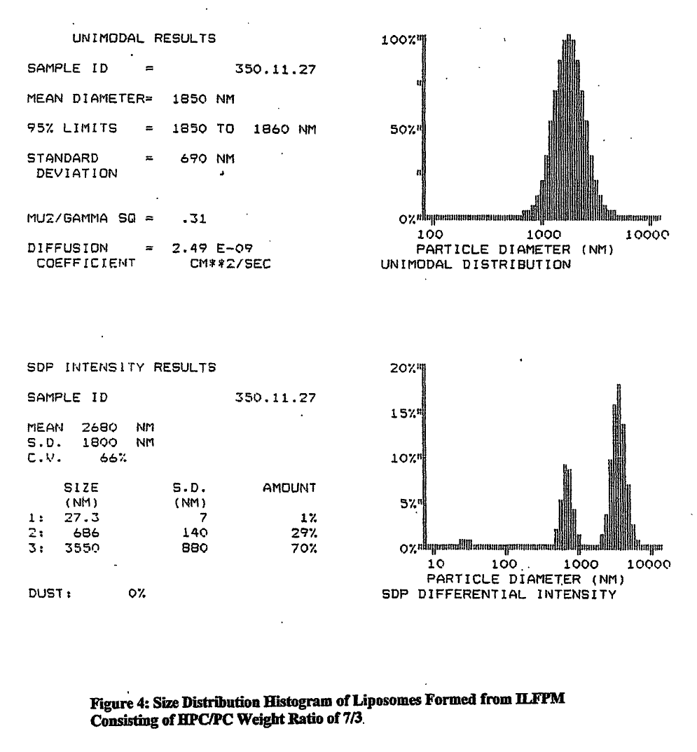

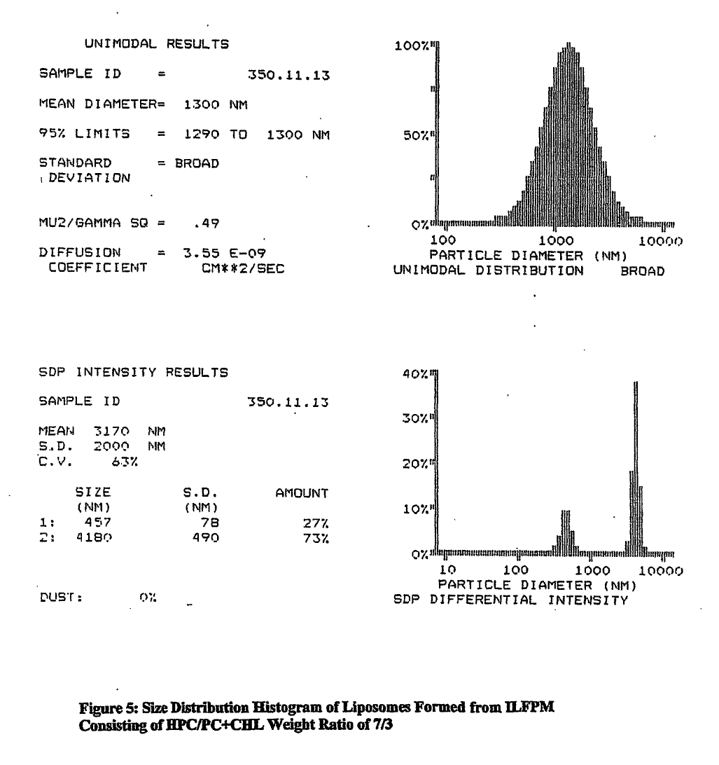

The size and size distribution analysis of ILFPM-based liposomes were performed using a submicron particle analyzer. The histograms illustrating the size distribution of the liposomes formed from HPC/PC (weight ratio of 7:3) and HPC/PC+cholesterol (weight ratio of 7:3) are presented in FIGS. 4 and 5 respectively. The liposomes received from both formulations showed unimodal distribution with mean diameter of 1850 nm and 1300 nm for the former and the latter formulations respectively. The SDP differential intensity results of both formulations showed, however, bimodal distribution. For instance the formulation which contained no cholesterol showed a large population of larger liposomes where most of the liposomes have diameter of 3550 nm and a smaller population of smaller liposomes having diameter of about 680 nm.

### 13.3. The Confocal Microscopy Results:

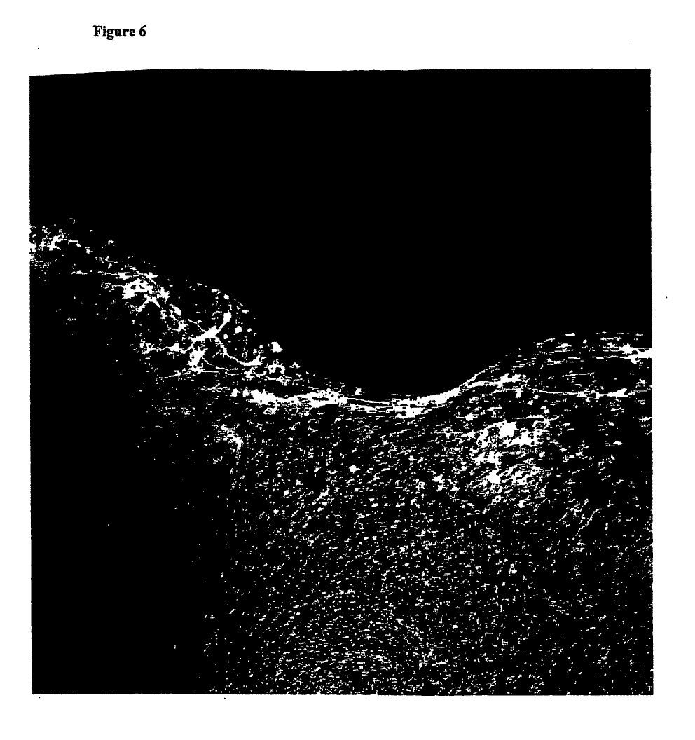

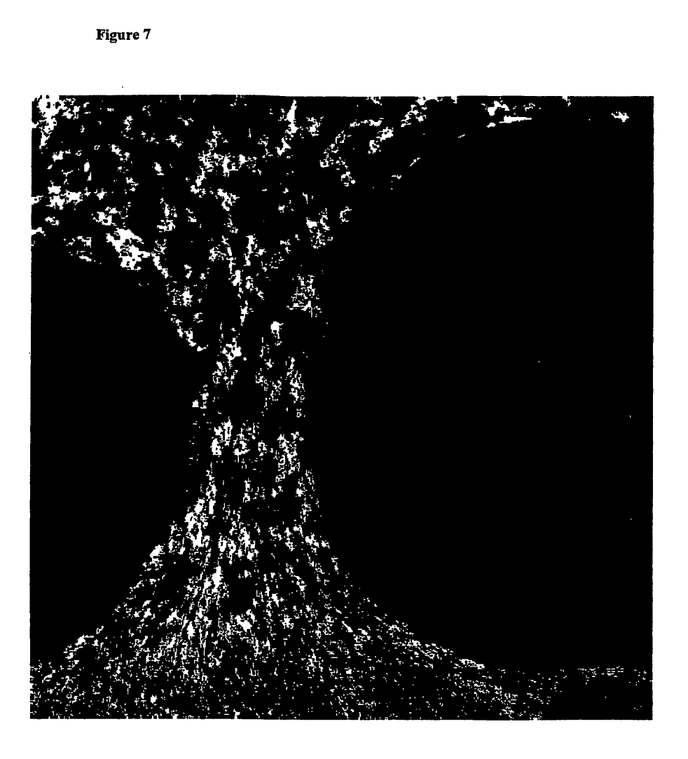

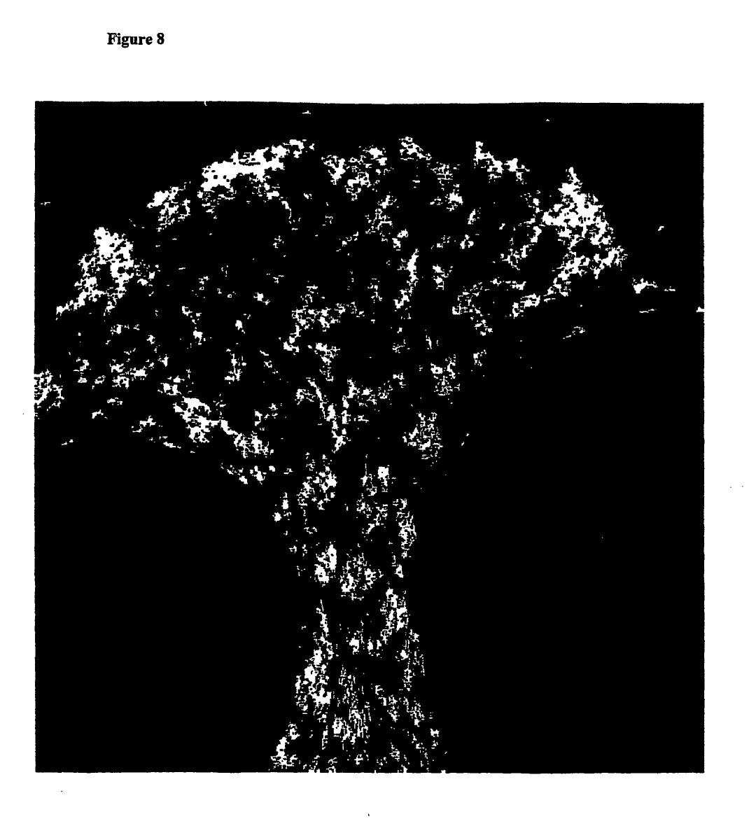

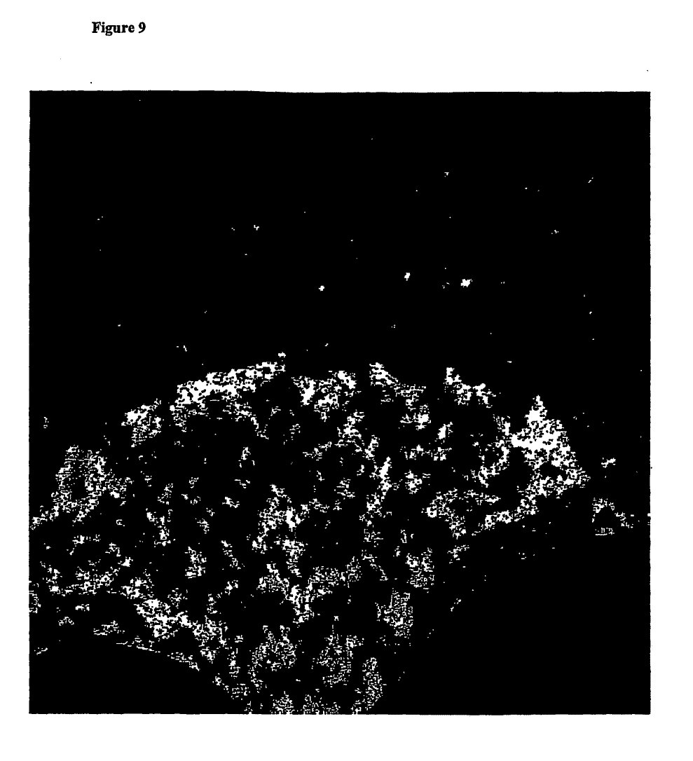

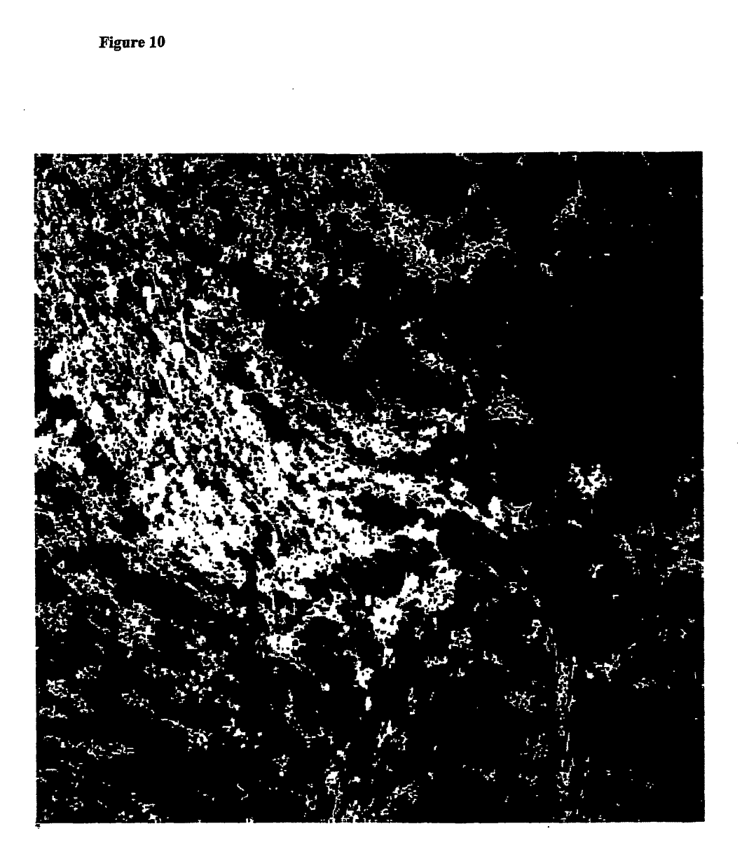

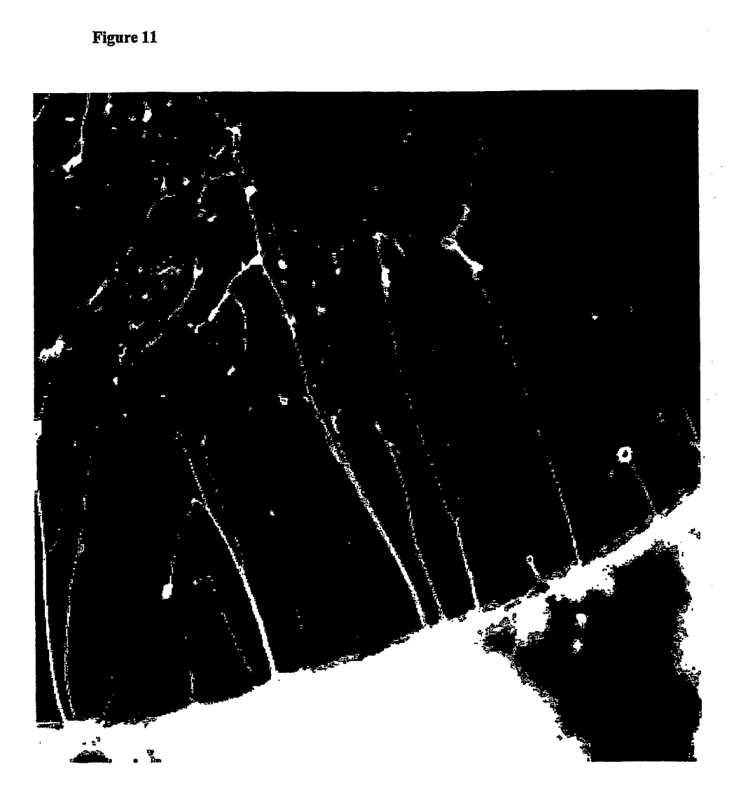

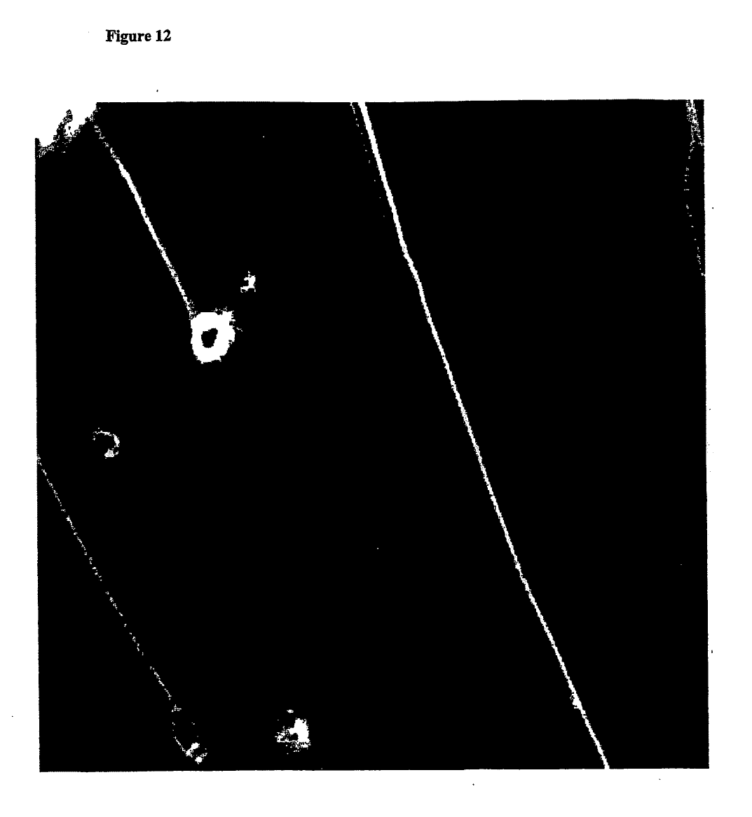

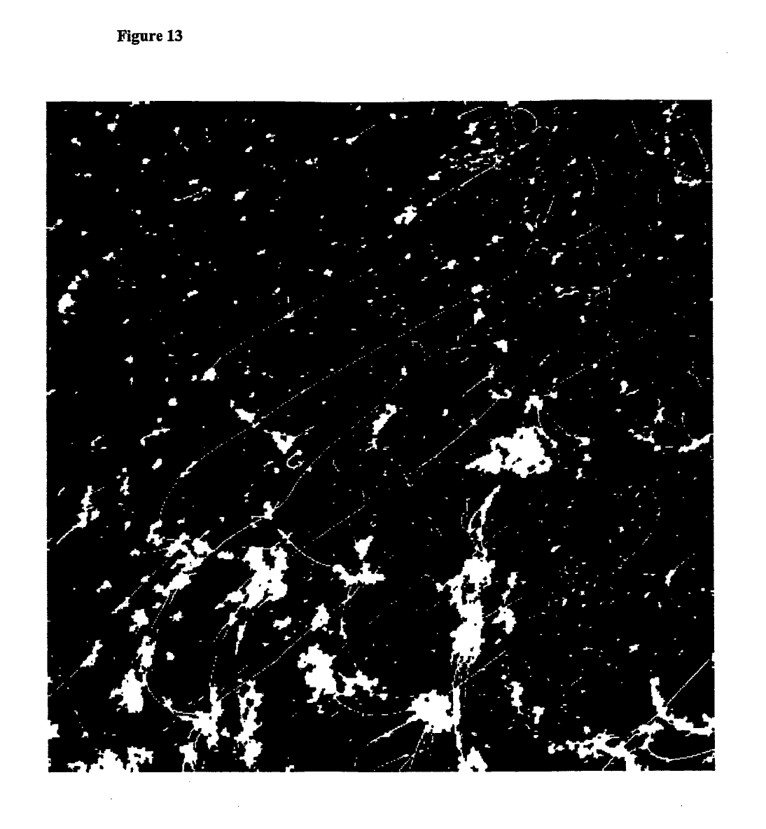

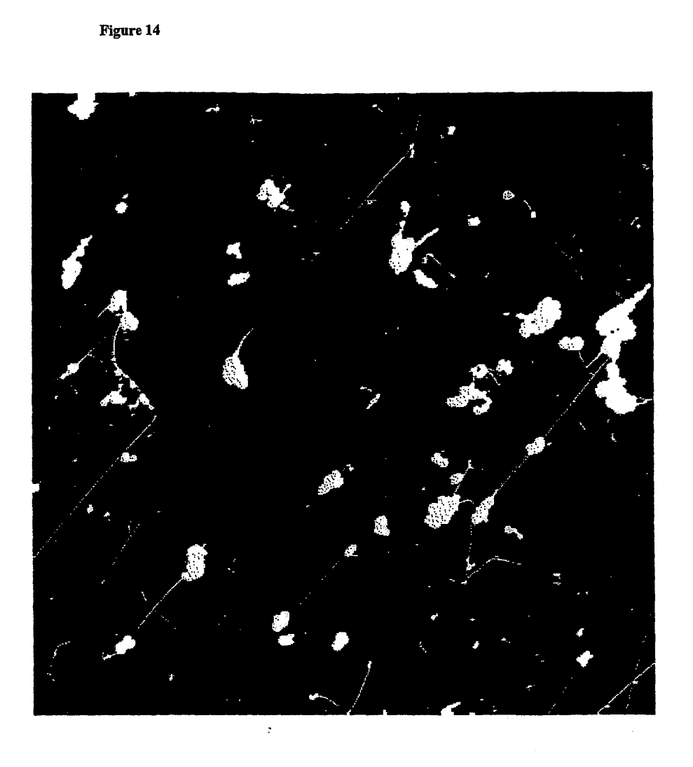

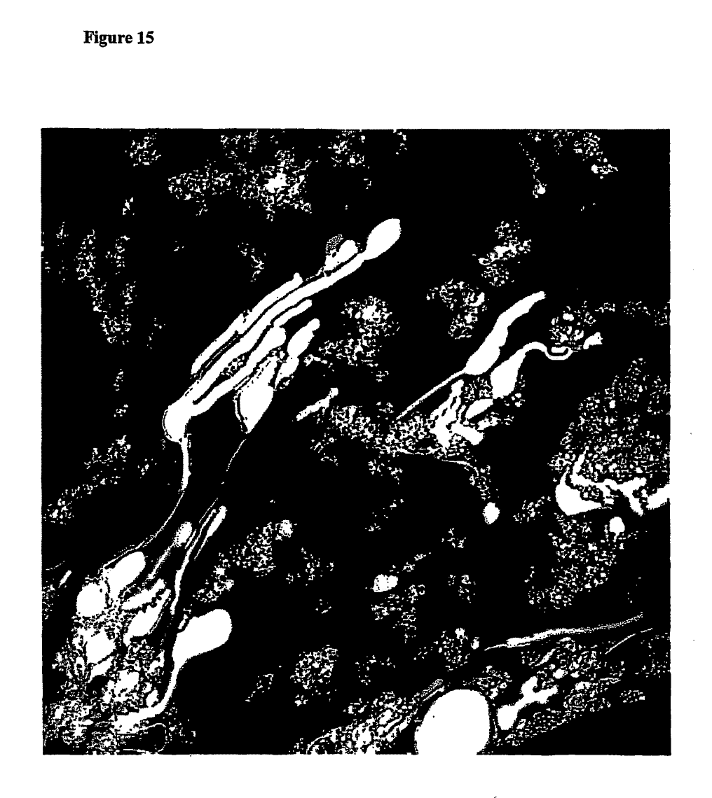

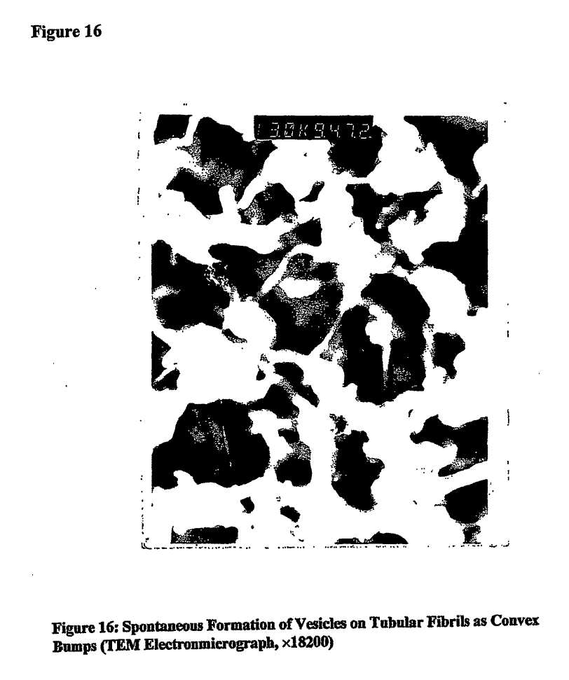

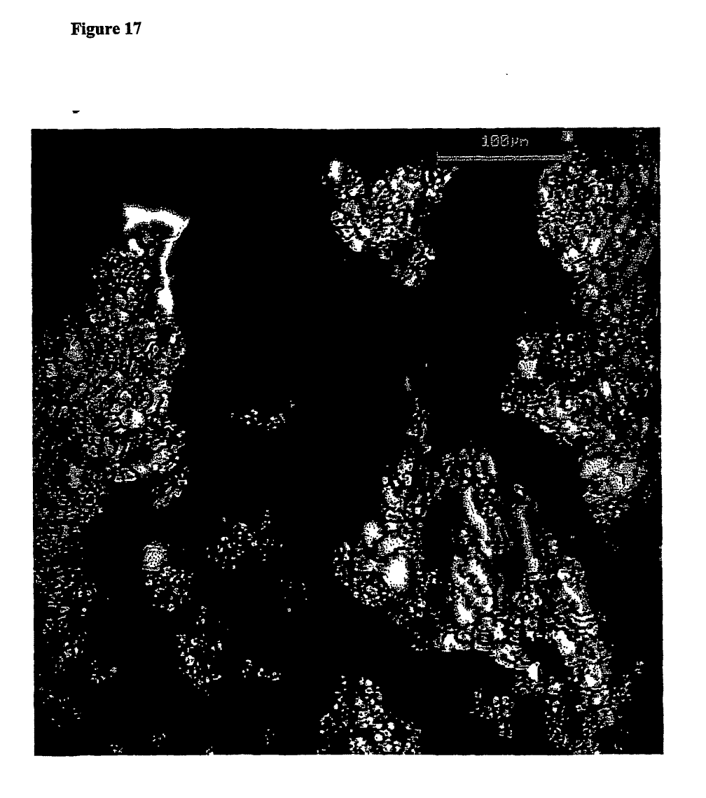

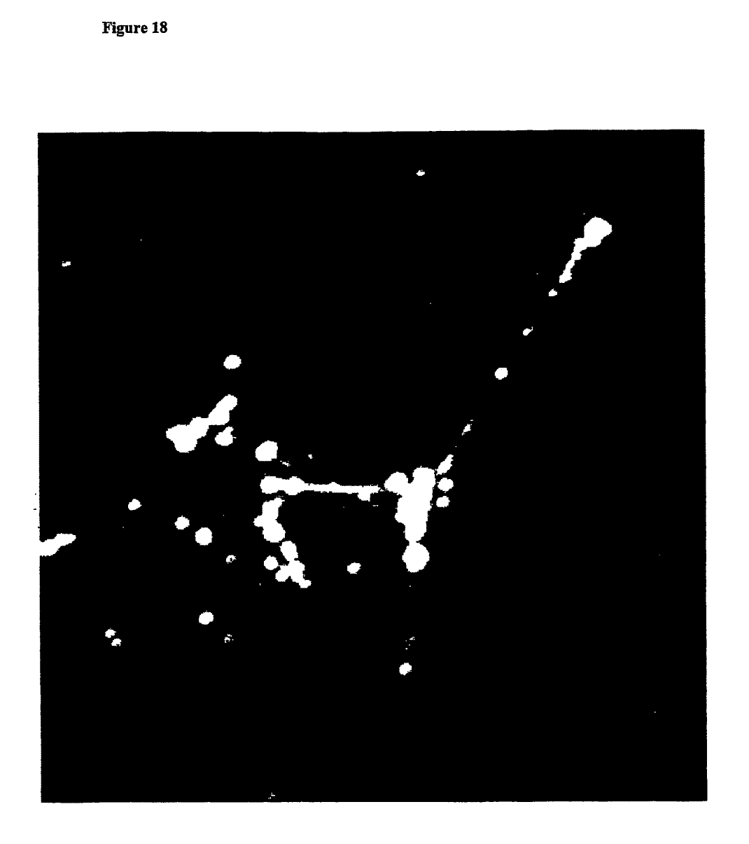

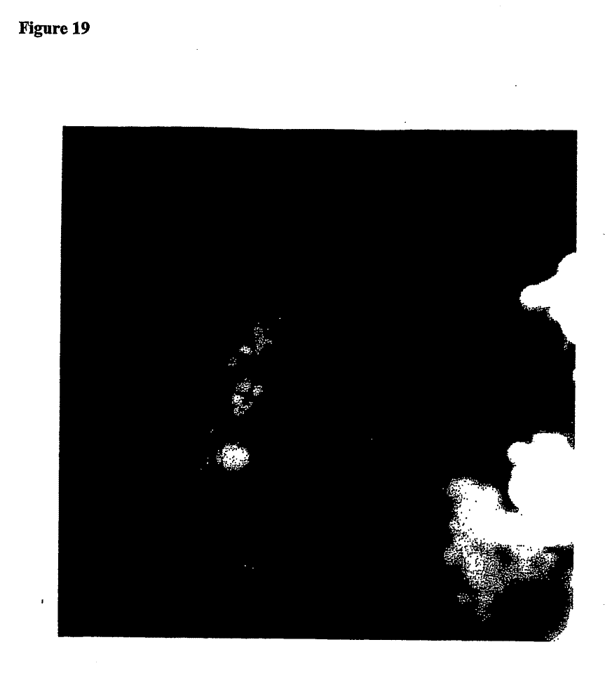

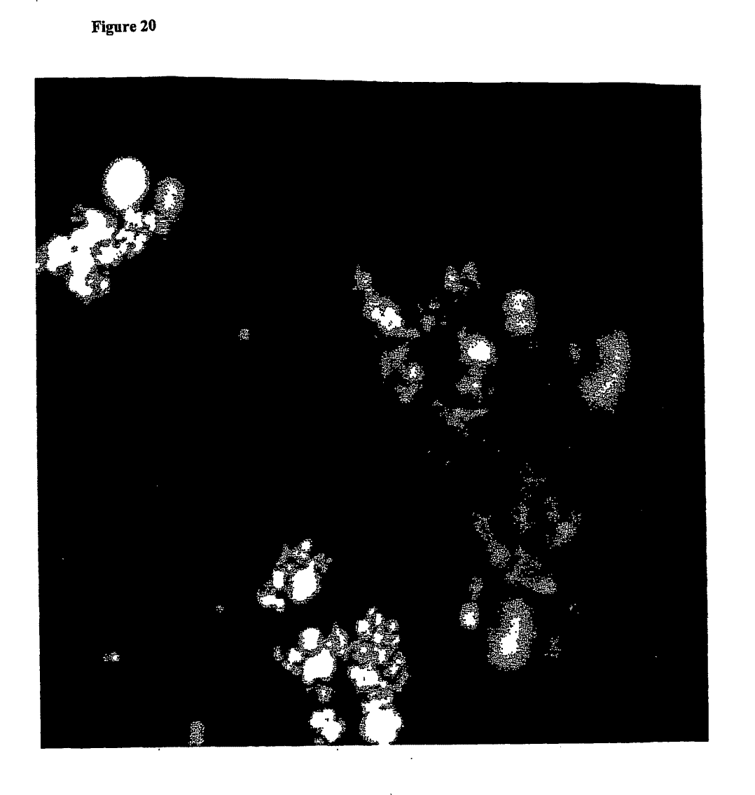

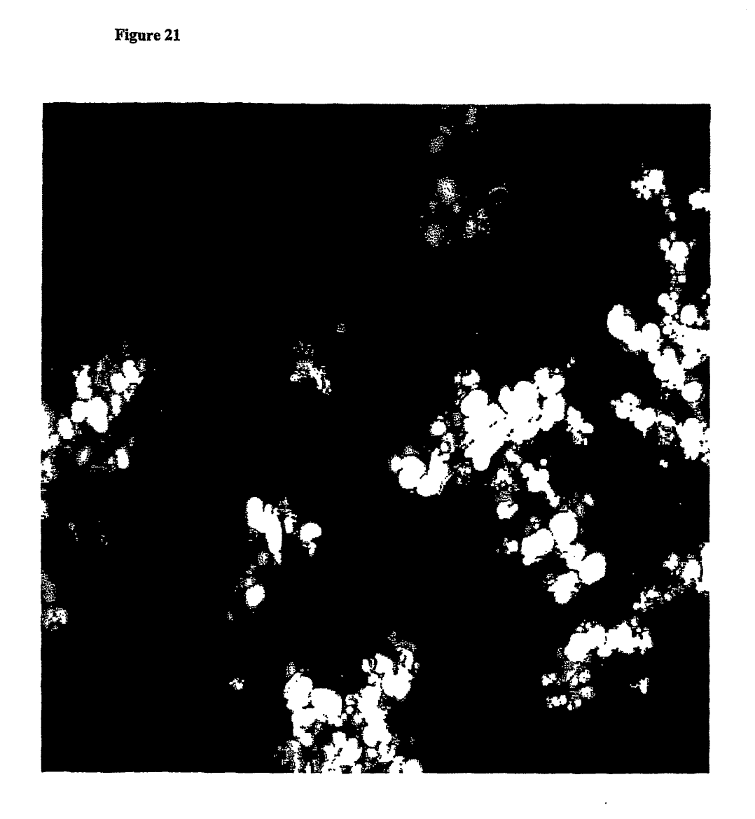

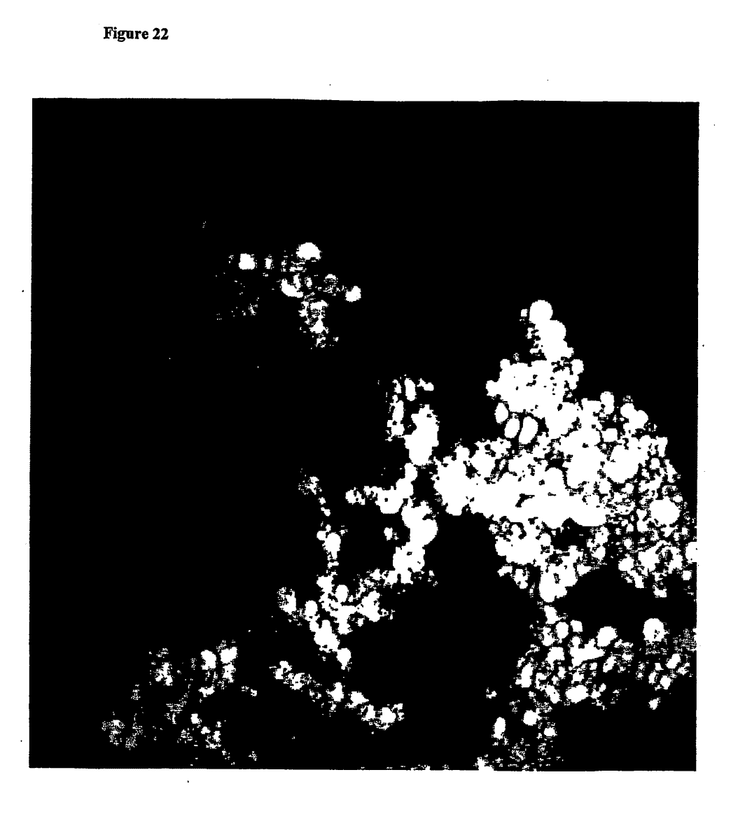

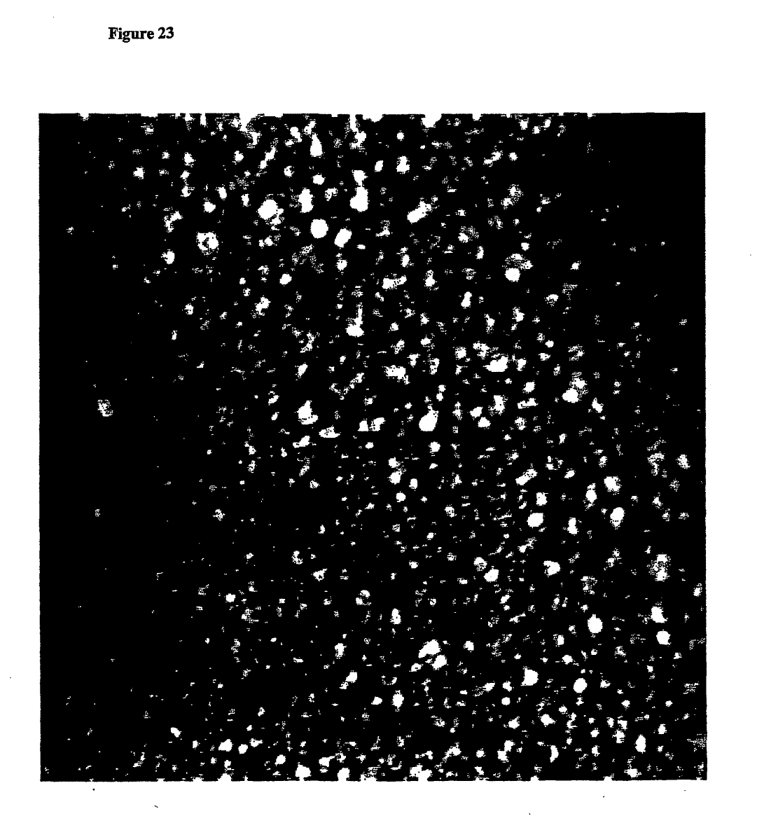

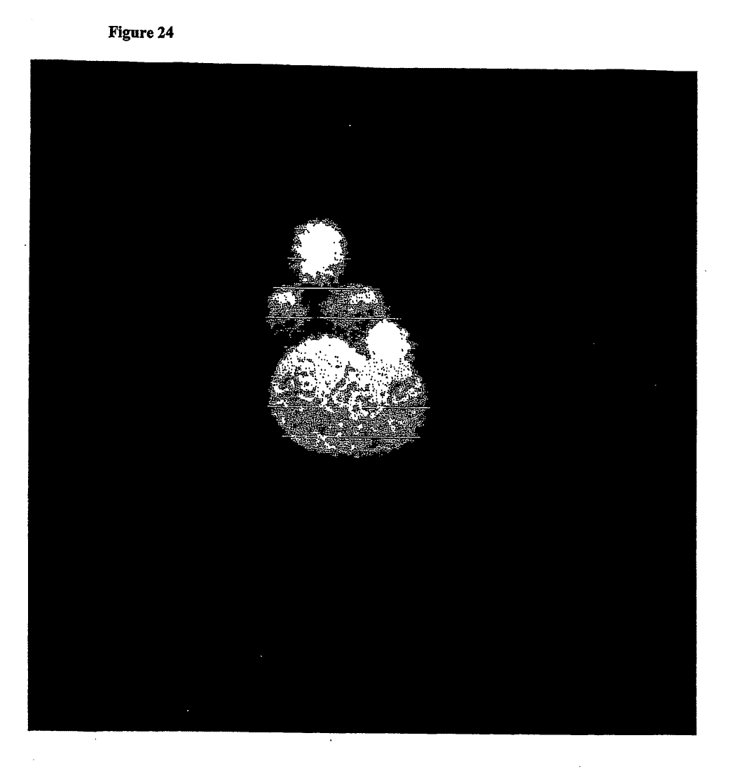

The confocal microscopy analysis was used to assess the mechanism of spontaneous vesicle formation from the ILFPM. It can be seen that upon the hydration of the system, first the dissolution of HPC takes place (FIGS. 6-10) followed by destruction of the film and finally vesiculation of liposomes via transformation of phospholipid to tubular fibril (FIGS. 11-23). Generally the first stage of the mechanism of vesicle formation is hydration of the phospholipid film. In the case of the conventional methods such as “thin lipid film” method by adding water to the dry phospholipid film the outer monolayer hydrates more than the inner ones. By contrast, it can be believed that the presence of a water soluble component such as HPC enhances the process of water penetration into the system by reducing both the interfacial tension between the aqueous medium and the lipid component, as well as the energy of the system and causes the system to increase its specific surface area (Saupe, A., J. Colloid Interface Sci., 58, 549-558, 1975) (FIGS. 6-10). This can be achieved due to the unique properties of HPC, or any other polymers alike, which was chosen carefully for this specific purpose. HPC is a surface-active polymer which can be compatible with surface active agents because of its hydroxypropyl substitution which imparts to the polymer some lipophilic nature. Water solutions of HPC display greatly reduced surface and interfacial tension. Therefore this unique property contributes significantly to the reduction of the interfacial tension between water and the phospholipid (as a mediator between two phases), consequently facilitates the wetting of ILFPM with no external energy dissipation. It is noteworthy that in most of the conventional liposome preparation methods some initial energy must be dissipated into the system in order to reduce the energy of the system and to enable the hydration of the system. The presence of HPC component in the formulation, therefore can save this energy dissipation. The reduction of the system energy causes the water to penetrate into the inner layers of the film and to form bilayers growing normally in the form of tubular fibrils which elongate (FIGS. 11-14). Hydrating bilayers are sliding into tubular fibrils, most likely in order to increase greatly the contact area with water where the polar heads can be exposed to water maximally. During this transformation the bilayers stabilize into their equilibrium distance which is a compromise between the repulsive undulation/steric and attractive van der Waals forces (Lasic, D. D., Biochem., J., 256, 1-11, 1988). It should be mentioned that the temperature at which the formation of the bilayer structure can be enabled must be higher than the gel to liquid crystalline phase transition temperature (Tc) of the phospholipid. Therefore the phospholipid chosen for spontaneous formation of liposomes should possess gel to crystalline phase transition temperature (Tc) lower than the body temperature (37° C.). In the further state upon the complete dissolution of HPC the tubular fibrils of bilayers separate from the matrix film. It is easy to see that in some spots depending on the state of the dissolution of HPC as well as the local crystallization defects, bunches of lamellae peel off (budding off) and they close to form vesicles (MLVs) (FIGS. 18-21). The vesicles are formed on these tubular fibrils as convex bumps (blisters) as a result of the penetration of water between the bilayers (FIGS. 15-17) and because the surface area of polar heads increases with the increasing hydration (Saupe, A., J. Colloid Interface Sci., 58, 549-558, 1975). Normally such a bunch of flakes blows up in its middle, disconnects from the surface and finally the lamellae close upon themselves to form groups of compact dispersed vesicles (FIGS. 22-23). With time the formed vesicles are released from the fibrils of phospholipid bilayers, and transfer to spherical form where their curvature energy is minimal and the entrapped volume maximal (FIGS. 23, 24). This is probably achieved via a directional flip-flop of phospholipid molecules because the number of molecules in the outside monolayer may be much larger than about one-half of all phospholipid molecules in the structure (Lasic, D. D., Biochem., J., 256, 1-11, 1988).

### Example 14

### Trapped Volume Analysis

The trapped volume (internal or capture volume) is expressed as the volume of the total internal aqueous compartment of the vesicle per unit quantity of lipid (1/mole lipid). In the present study this trapped volume is determined by entrapping a water soluble-marker such as 6-carboxyfluorescein (6-FAM) and measurement of trapped 6-FAM amount as described in the materials and methods section. The use of 6-FAM for this purpose was based on the fact that it can interact neither with lipids components nor the polymeric matrix (HPC). To prove that, solution of 6-FAM with a predetermined concentration was added to ILFPM containing no fluorescein marker. The concentration of the supernatant obtained after centrifugation of the suspension was found to be identical to that of the initial used marker solution.

The results of trapped volume demonstrated that the volume of the total internal aqueous compartment of in-situ resulted vesicle from ILFPM is 5 liters/mol PC. This value is higher about 4 and 5 fold than that of the MLV obtained spontaneously from pro-liposome method (European Patent 0158441) and than that of the MLV prepared by the “dry lipid-film hydration” method (Lichtenberg, D., Bahrenholz, Y., “Methods of Biological Analysis 33”, Glick, D. (Ed.), John Wiley&Sons, Inc., N.Y., 337461, 1988) respectively.

In the following examples the entrapment and entrapment efficiency of various active agents, as active material molecules models, possessing different water solubility in the spontaneously-formed liposomes were studied. Likewise the effects of other parameters such as; phospholipid content in the matrix, the weight ratio between phospholipid and either the drug or polymeric matrix, the volume of buffer used for hydration, the cholesterol content, the use of negatively-charged phospholipid, and the temperature of the hydrating medium on the entrapment and entrapment efficiency of Na-diclofenac were assessed.

### Example 15

### The Effect of PC Content in the Film

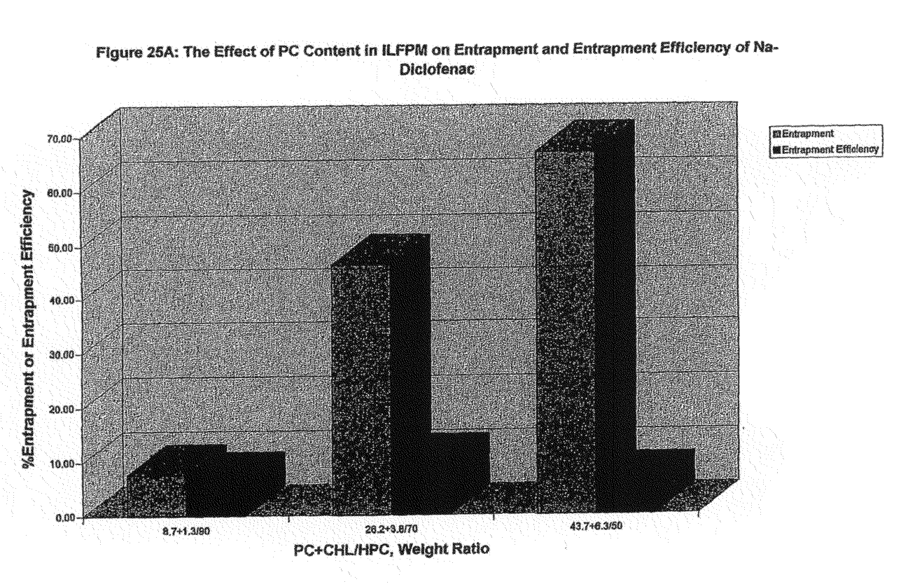

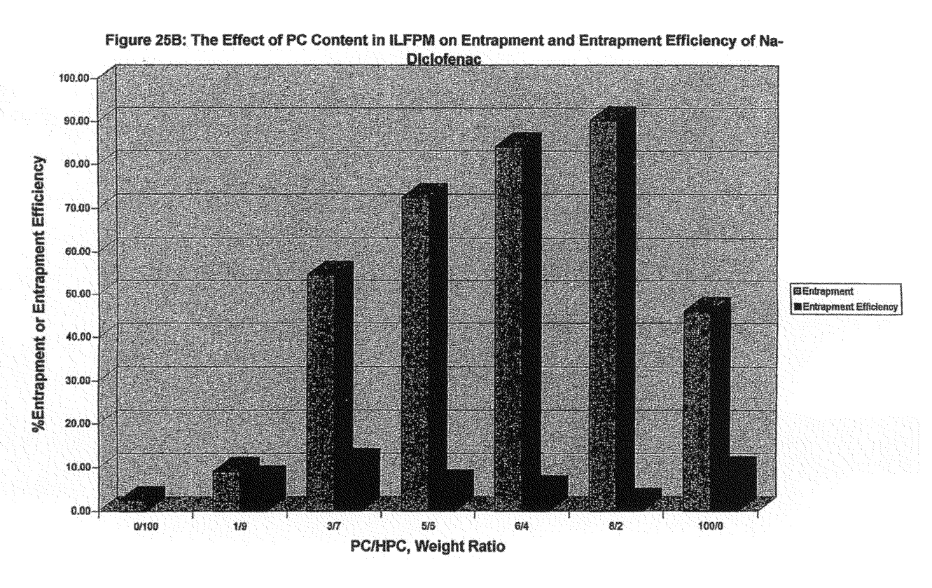

Films with various contents of PC were prepared where the content of the drug (sodium diclofenac) remained constant. The results of entrapment and entrapment efficiency of sodium diclofenac obtained from the formulations prepared for this purpose are summarized in table 3 and are also shown graphically in FIGS. 25A and 25B. The hydration of the films was carried out using 5 ml of intestinal fluid TS. The films were allowed to undergo dissolution by hand shaking for 5 minutes at room temperature. The results were compared to those received from the liposomes prepared using “modified thin lipid film” method and “classic thin lipid film” method as described in the materials and methods section. The result of the entrapment of Na-diclofenac in HPC film containing neither PC nor cholesterol is also shown in table 3.

It is demonstrated that the higher the content of phospholipid in the system the higher the entrapment of the active material. The highest entrapment efficiency, however, was received from the film which contained weight ratio of 3/7 of lipid/HPC. This film was also found to be mechanically the most stable and durable film as compared to the rest of the films.

The entrapment values obtained for all formulations are most likely the result of encapsulation of diclofenac in intraliposomal aqueous phase, although the interaction with the liposome by association with the bilayer components which can involve various forces such as electrostatic interaction, hydrophobic and Van der Waals can also contribute to the entrapment.

### Example 16

### The Effect of Active Material Content

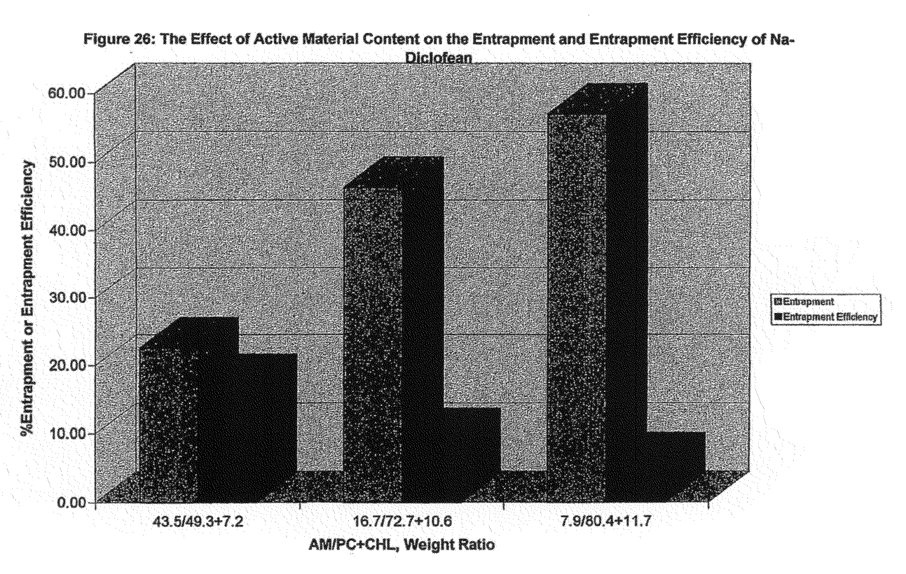

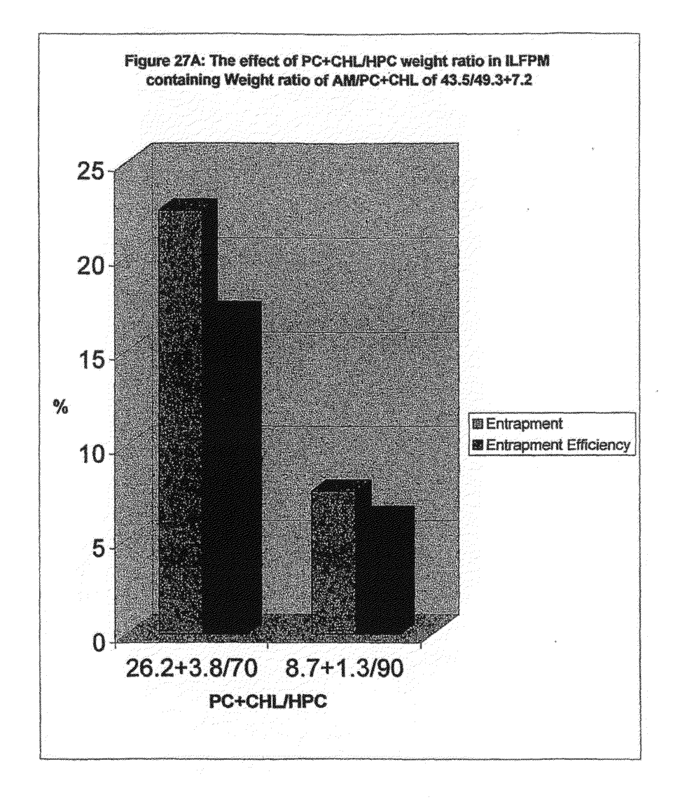

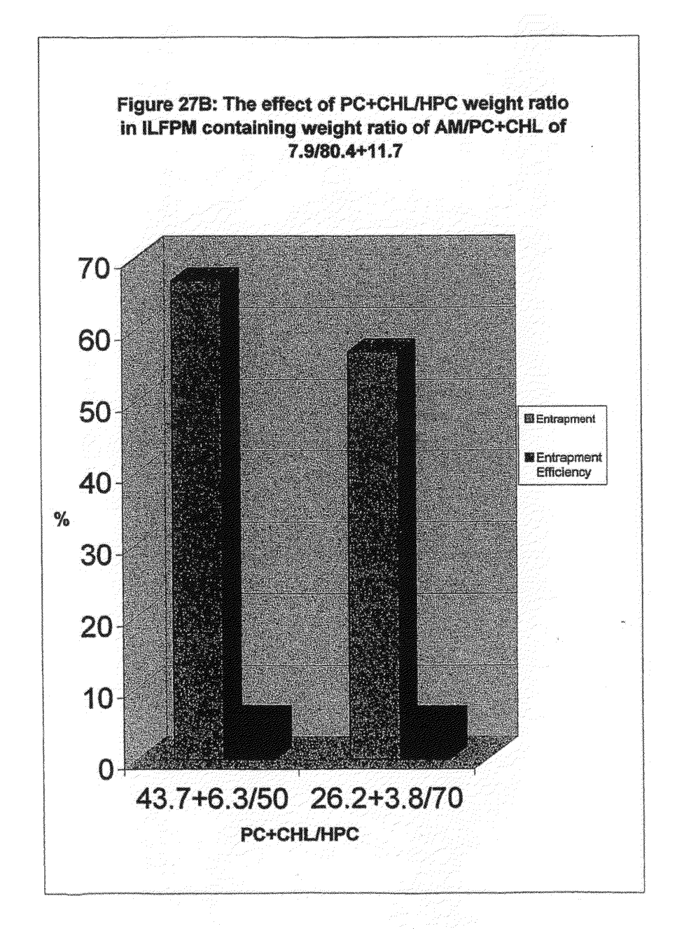

ILFPMs containing the same weight ratio of polymer to lipids but different active material contents were prepared and the entrapment and entrapment efficiency of Na-diclofenac were studied. The hydration of the films was carried out using 5 ml of intestinal fluid TS and the films were allowed to undergo dissolution by hand shaking for 5 minutes at room temperature. The results of % entrapment as well as entrapment efficiency are listed in table 4 and are illustrated in FIG. 26. It can be seen that with increasing the active material content in the formulation, % entrapment decreases and the entrapment efficiency increases. It is also interesting to see the comparison between the results of the entrapment from formulations possessing the same weight ratio of active material/lipid but different lipid/HPC (tables 3 and 4). This comparison is respectively shown in FIGS. 27A and 27B for two weight ratios of AM/lipid of 43.5/56.5 and 7.9/92.1. The figures show despite the identity in the weight ratio of AM/lipid the higher entrapment was resulted from the formulation containing higher weight ratio of lipid/HPC.

TABLE 3
The effect of PC content in ILFPM on entrapment and entrapment efficiency
(sodium diclofenac was used as a model of active material)

| | | PC + CHL/HPC | AM/HPC | AM/PC + CHL | Entrapment | Entrapment |
|---|---|---|---|---|---|---|
| Formulation | | weight ratio | weight ratio | weight ratio | w/w % | Efficien. % |
| W CHL | 349-35/6 | 8.7 + 1.3/90 | 7.9/92.1 | 43.5/49.3 + 7.2 | 7.6 | 6.2 |
| | 349-29/2 | 26.2 + 3.8/70 | 7.9/92.1 | 16.7/72.7 + 10.6 | 46.3 | 9.2 |
| | 349-35/10 | 43.7 + 6.3/50 | 7.9/92.1 | 7.9/80.4 + 11.7 | 66.9 | 5.7 |
| W/O CHL | 349-35/4 | 1/9 | 7.9/92.1 | 43.5/56.5 | 9 | 7.1 |
| | 349-42/2 | 3/7 | 7.9/92.1 | 16.7/83.3 | 54.5 | 11 |
| | 349-35/8 | 5/5 | 7.9/92.1 | 7.9/92.1 | 72.4 | 6.2 |
| | 349-87/9 | 6/4 | 7.9/92.1 | 5.4/94.6 | 84.2 | 4.8 |
| | 349-87/10 | 8/2 | 7.9/92.1 | 2.1/97.9 | 90.3 | 2 |
| MTLFM | 350-32/A | 100/0 | 100/0 | 16.7/83.3 | 46 | 9.2 |
| CTLFM | 285-127 | 100/0 | 100/0 | 16.7/83.3 | 51.8 | 10.4 |
| | 349-87/11 | 0/100 | 7.9/92.1 | | 2.4 | |

### Example 17

### The Effect of Cholesterol Content

In general the presence of cholesterol in the formulation is important since it reduces the sensitivity to osmotic rupture of the vesicles. Furthermore, the insertion of cholesterol into the egg PC bilayer reduces the leakage of the encapsulated drugs (Bahrenholz, Y., Crommelin, D. J., “Encyclopedia of Pharmaceutical Technology”, Swazbzick, J., Boylan, J. C., (Eds.), Marcel Dekker, N.Y., 1993). Addition of cholesterol to PC membranes has also a marginal effect on the position of the main transition temperature (Tc) (New, R. R. C. (Ed.), “Liposomes, A Practical Approach”, The Practical Approach Series, Series, Editors: D. Rickwood and B. D. Hames, Oris Press, 1997).

TABLE 4
The effect of active material content on the entrapment and entrapment
efficiency (sodium diclofenac was used as a model of active material)

| | PC + CHL/HPC | AM/HPC | AM/PC + CHL | Entrapment | Entrapment |
|---|---|---|---|---|---|
| Formulation | weight ratio | weight ratio | weight ratio | w/w % | Efficien. % |
| 349-44/1 | 26.2 + 3.8/70 | 24.8/75.2 | 43.5/49.3 + 7.2 | 22.5 | 17.1 |
| 349-29/2 | 26.2 + 3.8/70 | 7.9/92.1 | 16.7/72.7 + 10.6 | 46.3 | 9.2 |
| 349-44/2 | 26.2 + 3.8/70 | 3.6/96.4 | 7.9/80.4 + 11.7 | 57 | 5.6 |

Therefore, in the present invention various formulations differing in their cholesterol content were prepared and the effect of the presence of cholesterol on % encapsulation as well as entrapment efficiency of Na-diclofenac was assessed. These formulations are presented in table 5. The hydration of the films was carried out using 5 ml of intestinal fluid TS and the films were dissolved by hand shaking for 5 minutes at room temperature. The results of % encapsulation as well as entrapment efficiency are summarized in table 5.

TABLE 5
The effect of cholesterol on % entrapment of Na-diclofenac

| Formu- | Weight ratio | CHL/PC | Entrapment | Entrapment |
|---|---|---|---|---|
| lation | HPC:PC:CHL:AM | mole % | w/w % | Efficiency % |
| 349-42/2 | 66:28.3:0:5.7 | 0 | 54.5 | 11 |
| 349-47/7 | 66:24.8:1.5:5.7 | 10 | 51 | 11.1 |
| 349-29/2 | 66:24.7:3.6:5.7 | 22.2 | 46.4 | 9.2 |
| 349-47/8 | 66:23.2:5.1:5.7 | 30 | 42.8 | 8.6 |

One can see that with increasing the cholesterol content, a slight decrease in both entrapment as well as entrapment efficiency can occur. The decrease in the entrapment can be the result of the fact that cholesterol is added to formulation in place of PC and cholesterol does not by itself form bilayer structures. If it is not incorporated into the vesicle structure, the entrapment may be reduced following the reduction in PC/drug ratio. A further reason for this phenomenon may be the decrease in the vesicle size following the use of cholesterol.

### Example 18

### The Effect of Hydration Medium Volume

The spontaneous formation of liposomes can be initiated by exposing the ILFPM to an aqueous-based solution (hydrating medium). The liposome formation occurs simultaneously with the dissolution of HPC. The content of the hydration medium is determined by the oral cavity's unique environment. This aspect should be considered where the oral cavity is used for a drug delivery and drug absorption site.

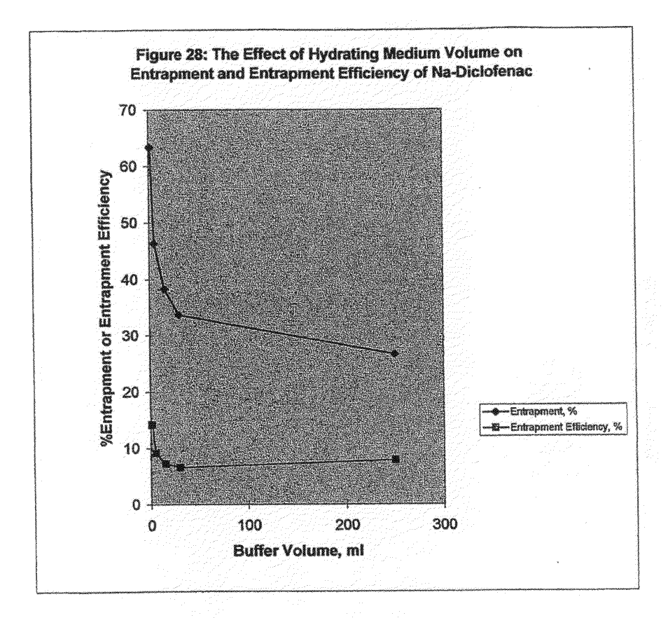

In the present examples buffer solution (intestinal fluid TS, pH=7.5) was used as hydrating medium as a model for saliva. Various amounts of the buffer solution were added to the ILFPM and after complete dissolution of the film by hand shaking at room temperature, the entrapment as well as the entrapment efficiency of Na-diclofenac were determined. The results are listed in table 6. ILFPMs consisting of weight ratio of 66:24.7:3.6:5.7 of HPC:PC:CHL:AM or 66:28.3:5.7 of HPC:PC:AM were used in all cases. The results are shown in FIG. 28. It should be mentioned that films containing HPC and active material, with no lipid components, resulted in an entrapment of 9.1% and 2.6%, according to theoretical content of diclofenac and that found in both precipitate and solution respectively. This higher value of the entrapment as compared to that appearing in table 3 (349-87/11) can be the result of entrapment of the active material in HPC gel resulting from the incomplete dissolution of HPC upon using a low volume of the buffer (1 ml). From the results one can obviously see that both % entrapment as well as entrapment efficiency increase with decreasing the hydrating medium volume. This is true for both formulations with and without cholesterol. The higher entrapment and entrapment efficiency resulting from using lower volume of hydrating medium can be ascribed to a lower leakage of Na-diclofenac from the inner liposome compartment upon dilution with the lower volume of the buffer. Likewise it can be the result of an efficient fusion of small vesicles during hydration with the lower volume of the buffer. Furthermore, the minimal volume of hydrating medium can reduce osmotic gradients and thus less osmotic rupture of the vesicles during the hydration process (Bahrenholz, Y., Crommelin, D. J., “Encyclopedia of Pharmaceutical Technology”, Swazbzick, J., Boylan, J. C., (Eds.), Marcel Dekker, N.Y., 1993). The minimal volume of hydrating medium can also result in a slower rate of dissolution process of the polymer which can result in more effective vesiculation of the liposomes as well as concentrating the solute near the phospholipid membranes during hydration.

TABLE 6
The effect of volume of hydrating medium on % entrapment and entrapment efficiency
of Na-diclofenac (the entrapment values are based on theoretical calculations)

| W CHL | | | | | W/O CHL | | | |
|---|---|---|---|---|---|---|---|---|
| Buffer | Buffer volume | | Entrapment | Entrapment | Buffer volume | | Entrapment | Entrapment |
| volume ml | ml/mg lipid | Formulation | % | Efficiency % | ml/mg PC | Formulation | % | Efficiency % |
| 1 | 0.08 | 349-35/2 | 63.4 | 14.2 | 0.08 | 349-42/2 | 73.7 | 17 |
| 5 | 0.4 | 349-29/2 | 46.4 | 9.2 | 0.39 | 349-42/2 | 54.5 | 11 |
| 15 | 1.22 | 349-29/2 | 38.3 | 7.3 | 1.2 | 349-42/2 | 41.1 | 8.3 |
| 30 | 2.45 | 349-35/2 | 33.7 | 6.6 | 2.49 | 349-42/2 | 34.7 | 7.0 |
| 250 | 29.3 | 349-72/2 | 26.6 | 7.9 | 25.1 | 349-72/1 | 39.4 | 5.4 |

This point constitutes a strong basis for the fact that the system according to the present invention can be effectually applied as a system for intra-oral delivery of insulin, since there is relatively little solvent into which the system can dissolve.

### Example 19

### The Use of Negatively-Charged Phospholipid and its Effect on Drug Loading

Generally the principle of use of negatively-charged phospholipid is based on the fact that the internal trap of neutral phospholipid MLVs can be increased by incorporating charged lipids into the membrane. This takes place by increasing the electrostatic repulsion between bilayers thus inducing swelling (Rand, R. P., Annu. Rev. Biophys. Bioeng., 10, 277-314, 1981, Gulik-Krzywicki, T., Rivas, E., Luzzati, V., J. Mol. Biol., 27, 303-322, 1967). Likewise including this kind of phospholipid in the formulation, causes improvement in physical stability of the liposomes by slowing down the process of aggregation and fusion (Lichtenberg, D., Bahrenholz, Y., “Methods of Biological Analysis 33”, Glick, D. (Ed.), John Wiley&Sons, Inc., N. Y., 337-461, 1988).

In the present study, however, the purpose of the use of negatively-charged phospholipid, phosphatidylserine (PS), was to improve the entrapment of the active materials. Sulindac, diclofenac, flurbiprofen and sodium salicylate, were used as active material models. Formulations (349-97/7, 349-9719, 349-97/11, 349-97/13, 349-97/14, 349-103/1) with weight ratio of 10% of PS/lipid were prepared and entrapment of the active materials was assessed. The entrapment, as well as entrapment efficiency of the active agents were determined by using 5 ml (for flurbiprofen 50 ml) of intestinal fluid TS (pH=7.5) as hydrating medium and by completing dissolution of the film by hand shaking at room temperature. The procedures of the hydration as well as the measurement of the entrapment were the same as described for the solubility effect of active material.

TABLE 7
Encapsulation of drugs in PS-containing ILFPM-formed liposomes

| Active | | | | | Entrapment |
|---|---|---|---|---|---|
| Material | Formulation | AM/Lipid % | AM/Film % | Entrapment % | Efficiency % |
| Sulindac | 349-97/11 | 20 | 5.7 | 6.3 | 1.3 |
| Diclofenac | 349-97/7 | 20 | 5.7 | 47.7 | 9.6 |
| Flurbiprofen | 349-103/1 | 5 | 1.5 | 19.8 | 1 |
| Flurbiprofen | ¹349-97/13 | 20 | 5.7 | 15.7 | 3.1 |
| Flurbiprofen | ²349-97/14 | 20 | 5.7 | 13.3 | 2.8 |
| Na-salicylate | 349-97/9 | 20 | 5.7 | 11.4 | 2.3 |

1. The weight ratio of PC+PS/HPC is 27+3/70 (see table 1)
2. The weight ratio of PC+PS/HPC is 28.5+1.5/70 (see table 1)

The results indicate that negatively-charged phospholipid appeared to have no effect on the entrapment of the active materials used (table 7).

### Example 20

### The Effect of Temperature on Entrapment

In these series of experiments the effect of temperature of the hydration process (the temperature of the hydrating medium) on the entrapment of Na-diclofenac (as a drug model) was assessed. The results are summarized in table 8.

The effect of temperature on entrapment is dependent on several variables such as the rate of the polymer dissolution, the rate of active material dissolution, partition coefficient of active material, the interaction between drug and phospholipid, the motion of fatty acid residues in the bilayers structure, the diffusion of drug from liposome (leakage) or into liposome, and the gel to liquid-crystalline phase transition (tₘ) of phospholipid. The phase change temperature of the various phospholipids is dependent on the chain length and the degree of saturation of the fatty acid components. The use of PC for ILFPM was based on the desire that the formation of the vesicles should be carried out spontaneously at physiological temperature and the fact that vesicles formation can be carried out only at a temperature which is above the gel to liquid crystal phase transition temperature of the phospholipid. Both egg PC as well as soybean PC are in a liquid crystal state at room temperature owing to their content of unsaturated fatty acids.

TABLE 8
The effect of temperature on entrapment of Na-diclofenac

| Temperature | | Formulation | Entrapment % |
|---|---|---|---|
| RT | W/O CHL | 349-42/2 | 54.5 |
| | W CHL | 349-29/2 | 46.4 |
| 37° C. | W/O CHL | 349-72/1 | 53.4 |
| | W CHL | 349-72/2 | 55.1 |
| 50° C. | W/O CHL | 349-97/1 | 49.4 |
| | W CHL | 349-97/2 | 43.0 |

The results show that the hydration at 37° C. resulted in an identical entrapment as obtained for the hydration which was carried out at room temperature (RT). With increasing the temperature to 50° C. a slight decrease in entrapment was received. This decrease in the entrapment can be the result of the effect of temperature on any parameter mentioned above.

### Example 21

### The Effect of Polymer Type

Polymers of different compositions were used to determine the effect of polymer on percent encapsulation of active material. For this purpose Na-diclofenac was used as active material model. The results of percent encapsulation using different polymers are summarized in table 9.

TABLE 9
Percent encapsulation of active material using different polymers

| | % Encapsulation of Active Material | | |
|---|---|---|---|
| | Formulations | Formulations | |
| Polymers content | with PC | w/o PC | Notes |
| Klucel LF + HF (95.1 + 4.9)% | 47.0 | 1.7 | LF—Low Molecular weight and viscosity |
| Klucel LF + HF (91.6 + 8.4)% | 49.9 | 1.8 | HF—High Molecular weight and viscosity |
| Klucel LF + HF (80.0 + 20.0)% | 48.3 | 7.5 | |
| Klucel LF + HF (70.0 + 30.0)% | 46.2 | 0.2 | |
| Klucel LF + EC 20 (95.1 + 4.9)% | 55.8 | 0.8 | EC 20—Ethylcellulose 20 |
| Klucel LF + EC 20 (9.16 + 8.4)% | 54.4 | 5.2 | |
| Klucel LF + EC 20 (80.0 + 20.0)% | 57.7 | 5.9 | |
| Klucel LF + EC 20 (70.0 + 30.0)% | 53.8 | 10.9 | |
| Klucel LF (100%) | 54.5 | 2.3 | |
| Polyacrylic Acid (100%) | 90.0 | 84.5 | |
| Polyacrilic Acid + PEG 600 (70.0 + 30.0)% | 78.5 | 73.3 | PEG 600—Poyethylene Glycol 600 |
| Plasdone K-29-32 (100%) | 30.7 | | |
| Kolidone K90 + PEG 600 (70.0 + 30.0)% | 39.0 | | |
| Kollidone VA 64 (100%) | 36.5 | | |

### Example 22

### Entrapment of Polymeric Matrix

The entrapment of HPC in the spontaneously formed liposomes was assessed using gel permeation chromatography method. The HPC entrapment was determined by determining the amount of HPC in the precipitate obtained after centrifugation of the suspension obtained from the hydration of ILFPM, as it was mentioned in the section of “materials and methods”. The results of the entrapment of HPC from various formulations used for ILFPM preparation are listed in table 10.

TABLE 10
The entrapment of HPC in spontaneously
formed liposomes from ILFPM

| | HPC/PC | AM | CHL | Encapsulation, |
|---|---|---|---|---|
| Formulation | (W/W) | Content¹ | Content | % |
| 349-35/3 | 90 | W/O AM | W/O CHL | 0.7 |
| 349-35/5 | | W/O AM | W CHL | 0.6 |
| 349-47/9 | 70 | W/O AM | W/O CHL | 1.7 |
| 349-47/10 | | W/O AM | W CHL | 1.5 |
| 349-35/7 | 30 | W/O AM | W/O CHL | 2.7 |
| 349-35/9 | | W/O AM | W CHL | 4.4 |
| 349-35/4 | 90 | W AM | W/O CHL | 1.1 |
| 349-35/6 | | W AM | W CHL | 0.7 |
| 349-72/1 | 70 | W AM | W/O CHL | 1.1 |
| 349-72/2 | | W AM | W CHL | 2.8 |
| 349-35/8 | 30 | W AM | W/O CHL | 2.2 |
| 349-35/10 | | W AM | W CHL | 1.0 |

¹Na-diclofenac was used as active material

As it can be seen the encapsulation of HPC during the spontaneous formation of liposomes is negligible. This finding confirms the fact that beyond the solubility characteristics of solute which affects the entrapment significantly, the molecular weight of the agent can play an important role as well. It can be also concluded that no interaction exists between HPC and PC. The results demonstrate also that no significant effect of either active materials or CHL on the encapsulation of HPC was found since no significant difference between HPC encapsulations were obtained from different formulations used for preparation of ILFPMs.

### Example 23

1. Sodium lauryl sulphate and acesulfame potassium was added into insulin solution.
2. Menthol, peppermint oil and m-cresol were dissolved in ethanol.
3. After completely dissolution of the components, the solutions were mixed together to which hydroxypropyl cellulose and lecithin were added.
4. The resulting mixture (after completely dissolution of all components) was poured into a mold and allowed to be dried at room temperature to receive a solid film.

TABLE 11
The composition of Example 23

| Materials | g/batch |
|---|---|
| Insulin solution | 384.5 |
| Ethanol | 279.7 |
| Hydroxypropyl Cellulose | 32.6 |
| Lecithin | 13.9 |
| Sodium Lauryl Sulphate | 6.4 |
| Acesulfame Potassium | 0.4 |
| Menthol | 1.5 |
| Peppermint oil | 0.3 |
| m-cresol | 0.5 |
| Total | 720.0 |

### Example 24

1. Emulgin LM-23 and then Insulin were dissolved in saline phosphate buffer pH 7.4. For improving dissolution, water purified was added.
2. Menthol, peppermint oil and m-cresol were dissolved in ethanol.
3. After completely dissolution of the components, the solutions were mixed together to which hydroxypropyl cellulose, lecithin and acesulfame potassium were added.
4. The resulting mixture (after completely dissolution of all components) was poured into a mold and allowed to be dried at room temperature to receive a solid film.

TABLE 12
The composition of Example 24

| Materials | g/batch |
|---|---|
| Insulin | 0.04 |
| Buffer pH 7.4 | 9.32 |
| Emulgin LM 23 | 0.45 |
| Water purified | 1.01 |
| Ethanol | 6.72 |
| Hydroxypropyl Cellulose | 0.79 |
| Lecithin | 0.34 |
| Menthol | 0.49 |
| Peppermint oil | 0.02 |
| m-cresol | 0.01 |
| Acesulfame potassium | 0.01 |
| Total | 19.19 |

### Example 25

1. Sodium lauryl sulphate and then Insulin were dissolved in saline phosphate buffer pH 7.4.
2. Menthol, peppermint oil, m-cresol and emulgin LM-23 were dissolved in ethanol.
3. After completely dissolution of the components, the solutions were mixed together to which hydroxypropyl cellulose, lecithin and acesulfame potassium were added.
4. The resulting mixture (after completely dissolution of all components) was poured into a mold and allowed to be dried at room temperature to receive a solid film.

TABLE 13
The composition of Example 25

| Materials | g/batch |
|---|---|
| Insulin | 0.04 |
| Buffer pH 7.4 | 9.31 |
| Emulgin LM 23 | 0.45 |
| Sodium Lauryl Sulphate | 0.16 |
| Ethanol | 6.80 |
| Hydroxypropyl Cellulose | 0.79 |
| Lecithin | 0.34 |
| Menthol | 0.05 |
| Peppermint oil | 0.01 |
| m-cresol | 0.02 |
| Acesulfame potassium | 0.13 |
| Total | 18.10 |

### Example 26

1. Sodium lauryl sulphate and then Insulin were dissolved in saline phosphate buffer pH 7.4.
2. Menthol, peppermint oil, m-cresol, emulgin LM-23 and lecithin were dissolved in ethanol.
3. After completely dissolution of the components, the solutions were mixed together to which carbopol, hydroxypropyl cellulose and acesulfame potassium were added.
4. The resulting mixture (after completely dissolution of all components) was poured into a mold and allowed to be dried at room temperature to receive a solid film.

TABLE 14
The composition of Example 26

| Materials | g/batch |
|---|---|
| Insulin | 0.04 |
| Buffer pH 7.4 | 9.33 |
| Emulgin LM 23 | 0.45 |
| Sodium Lauryl Sulphate | 0.16 |
| Ethanol | 6.82 |
| Hydroxypropyl Cellulose | 0.40 |
| Carbopol | 0.15 |
| Lecithin | 0.34 |
| Menthol | 0.06 |
| Peppermint oil | 0.01 |
| m-cresol | 0.02 |
| Acesulfame potassium | 0.02 |
| Total | 17.48 |

### Example 27

1. Sodium laury sulphate, sodium salicylate and then Insulin were added into saline phosphate buffer pH 7.4.
2. Menthol, peppermint oil, m-cresol, and lecithin were dissolved in ethanol.
3. After completely dissoluton of the components, the solutions were mixed together to which hydroxypropyl cellulose and acesulfame potassium were added.
4. The resulting mixture (after completely dissolution of all components) was poured into a mold and allowed to be dried at room temperature to receive a solid film.

TABLE 15
The composition of Example 27

| Materials | g/batch |
|---|---|
| Insulin | 0.04 |
| Buffer pH 7.4 | 9.32 |
| Sodium Lauryl Sulphate | 0.50 |
| Sodium Salicylate | 0.50 |
| Ethanol | 6.82 |
| Hydroxypropyl Cellulose | 0.79 |
| Lecithin | 0.34 |
| Menthol | 0.05 |
| Peppermint oil | 0.01 |
| m-cresol | 0.02 |
| Acesulfame potassium | 0.05 |
| Total | 18.11 |

### Example 28

1 Insulin. and then sodium laury sulphate were added into saline phosphate buffer pH 7.4.
2. Menthol, peppermint oil, m-cresol, lecithin and capric acid were dissolved in ethanol.
3. After completely dissolution of the components, the solutions were mixed together to which hydroxypropyl cellulose and acesulfame potassium were added.
4. The resulting mixture (after completely dissolution of all components) was poured into a mold and allowed to be dried at room temperature to receive a solid film.

TABLE 16
The composition of Example 28

| Materials | g/batch | g/batch |
|---|---|---|
| Insulin | 0.04 | 0.04 |
| Buffer pH 7.4 | 9.34 | 9.31 |
| Sodium Lauryl Sulphate | 0.16 | 0.50 |
| Ethanol | 6.80 | 6.81 |
| Hydroxypropyl Cellulose | 0.79 | 0.79 |
| Capric acid | 0.08 | 0.09 |
| Lecithin | 0.34 | 0.34 |
| Menthol | 0.05 | 0.05 |
| Peppermint oil | 0.01 | 0.01 |
| m-cresol | 0.02 | 0.02 |
| Acesulfame potassium | 0.05 | 0.05 |
| Total | 17.66 | 18.00 |

### Examples 29a, 29b, 29c

1. Sodium laury sulphate (a) or sodium salicylate (b) or EDTA tetrasodium salt (c) were dissolved in saline phosphate buffer pH 7.4 and then Insulin was added.
2. Menthol, peppermint oil and m-cresol, were dissolved in ethanol.
3. After completely dissolution of the components, the solutions were mixed together to which hydroxypropyl cellulose and acesulfame potassium were added.
4. The resulting mixture (after completely dissolution of all components) was poured into a mold and allowed to be dried at room temperature to receive a solid film.

TABLE 17
The composition of Example 29

| Materials | g/batch | g/batch | g/batch |
|---|---|---|---|
| Insulin | 0.04 | 0.04 | 0.04 |
| Buffer pH 7.4 | 9.32 | 9.31 | 9.31 |
| Emulgin LM-23 | | 0.50 | |
| Sodium Lauryl Sulphate | 0.50 | | |
| EDTA Tetrasodium | | | 0.25 |
| Ethanol | 6.80 | 6.83 | 7.16 |
| Hydroxypropyl Cellulose | 0.79 | 0.79 | 0.79 |
| Menthol | 0.05 | 0.05 | 0.04 |
| Peppermint oil | 0.01 | 0.01 | 0.01 |
| m-cresol | 0.02 | 0.02 | 0.02 |
| Acesulfame potassium | 0.04 | 0.04 | 0.04 |
| Total | 17.59 | 17.55 | 17.66 |

### Example 30

1. Insulin, sodium laury sulphate, and then beta-cyclodextrin. were added into saline phosphate buffer pH 7.4.
2. Menthol, peppermint oil, m-cresol and lecithin were dissolved in ethanol.
3. After completely dissolution of the components, the solutions were mixed together to which hydroxypropyl cellulose and asesulfame potassium were added.
4. The resulting mixture (after completely dissolution of all components) was poured into a mold and allowed to be dried at room temperature to receive a solid film.

TABLE 18
The composition of Example 30

| Materials | g/batch | g/batch |
|---|---|---|
| Insulin | 0.08 | 0.09 |
| Buffer pH 7.4 | 23.26 | 23.28 |
| Sodium Lauryl Sulphate | 0.40 | 0.40 |
| Beta-Cyclodextrln hydrate | 0.20 | 0.40 |
| Ethanol | 17.04 | 17.03 |
| Hydroxypropyl Cellulose | 1.98 | 1.98 |
| Lecithin | 0.85 | 0.85 |
| Menthol | 0.11 | 0.10 |
| Peppermint oil | 0.03 | 0.03 |
| m-cresol | 0.04 | 0.05 |
| Acesulfame potassium | 0.03 | 0.03 |
| Total | 44.01 | 44.23 |

### Example 31

1. Sodium laury sulphate was added into insulin solution.
2. Menthol, peppermint oil and m-cresol were dissolved in ethanol.
3. After completely dissolution of the components, the solutions were mixed together to which laureth-9, lecithin, hydroxypropyl cellulose and acesulfame potassium were added.
4. The resulting mixture (after completely dissolution of all components) was poured into a mold and allowed to be dried at room temperature to receive a solid film.

TABLE 19
The composition of Example 31

| Materials | g/batch | g/batch |
|---|---|---|
| Insulin solution | 4.68 | 4.65 |
| Sodium Lauryll Sulphate | 0.07 | 0.07 |
| Laureth-9 | 0.40 | 0.20 |
| Ethanol | 3.15 | 3.18 |
| Hydroxypropyl Cellulose | 0.37 | 0.37 |
| Lecithin | 0.16 | 0.16 |
| Menthol | 0.02 | 0.02 |
| Peppermint oil | 0.004 | 0.004 |
| m-cresol | 0.009 | 0.009 |
| Acesulfame potassium | 0.02 | 0.02 |
| Total | 8.903 | 8.683 |

### Example 32

1. Sodium lauryl sulphate and sodium glycodeoxycholate were added into insulin solution.
2. Menthol, peppermint oil and m-cresol were dissolved in ethanol.
3. After completely dissolution of the components, the solutions were mixed together to which lecithin, hydroxypropyl cellulose and acesulfame potassium were added.
4. The resulting mixture (after completely dissolution of all components) was poured into a mold and allowed to be dried at room temperature to receive a solid film.

TABLE 20
The composition of Example 32

| Materials | g/batch |
|---|---|
| Insulin solution | 5.01 |
| Sodium Lauryll Sulphate | 0.08 |
| Sodium Glycodeoxycholate | 0.12 |
| Ethanol | 3.41 |
| Hydroxypropyl Cellulose | 0.40 |
| Lecithin | 0.17 |
| Menthol | 0.02 |
| Peppermint oil | 0.005 |
| m-cresol | 0.009 |
| Acesulfame potassium | 0.02 |
| Total | 9.244 |

### Example 33

1. Sodium lauryl sulphate and different type of cyclodextrin were added into insulin solution.
2. Menthol, peppermint oil and m-cresol were dissolved in ethanol.
3. After completely dissolution of the components, the solutions were mixed together to which lecithin, hydroxypropyl cellulose and acesulfame potassium were added.
4. The resulting mixture (after completely dissolution of all components) was poured into a mold and allowed to be dried at room temperature to receive a solid film.

TABLE 21
The composition of Example 33

| Materials | g/batch | g/batch | g/batch |
|---|---|---|---|
| Insulin solution | 5.49 | 5.03 | 5.07 |
| Sodium Lauryll Sulphate | 0.09 | 0.08 | 0.08 |
| Alfa-Cyclodextrin | 0.05 | | |
| Beta-Cyclodextrin | | 0.04 | |
| Gamma-Cyclodextrin | | | 0.04 |
| Ethanol | 3.65 | 3.40 | 3.40 |
| Hydroxypropyl Cellulose | 0.43 | 0.43 | 0.43 |
| Lecithin | 0.19 | 0.17 | 0.17 |
| Menthol | 0.02 | 0.02 | 0.02 |
| Peppermint oil | 0.005 | 0.005 | 0.005 |
| m-cresol | 0.009 | 0.008 | 0.008 |
| Acesulfame potassium | 0.02 | 0.02 | 0.02 |
| Total | 9.954 | | |

### Example 34

1. Sodium lauryl sulphate and beta-cyclodextrin were added into insulin solution.
2. Menthol, peppermint oil and m-cresol were dissolved in ethanol.
3. After completely dissolution of the components, the solutions were mixed together to which lecithin, hydroxypropyl cellulose, lecithin, and acesulfame potassium were added.
4. The resulting mixture (after completely dissolution of all components) was poured into a mold and allowed to be dried at room temperature to receive a solid film.

TABLE 22
The composition of Example 34

| Materials | g/batch | g/batch |
|---|---|---|
| Insulin solution | 12.01 | 12.00 |
| Sodium Lauryll Sulphate | 0.19 | 0.19 |
| Beta-Cyclodextrin | 0.19 | 0.19 |
| Ethanol | 8.16 | 8.29 |
| Hydroxypropyl Cellulose | 0.95 | 0.95 |
| Lecithin | 0.41 | 0.48 |
| Menthol | 0.05 | 0.05 |
| Peppermint oil | 0.01 | 0.01 |
| m-cresol | 0.02 | 0.02 |
| Acesulfame potassium | 0.04 | 0.04 |
| Total | 22.03 | 22.22 |

### Materials:

Human Insulin-Bulk (r-DNA origin), Biocon, India Lot B-040319A
Insulin solution-Humulin, Lilly France, Lot FF 4J84A and FF5G39C
Hydroxypropyl Cellulose, Hercules, Belgium Lot 8932
Lecithin (Epicuron 200), Degussa Germany Lot 1-3-9065
Carbopol 71G, Goodrich, Belgium Lot CTO75GJ012
Sodium Lauryl Sulphate, Cognis, Germany Lot CS20650014
Sodium Salicylate, MERCK, Lot F637302428
EDTA Tetrasodium, Sigma Lot 1247-0296
Capric acid, Fluka Lot RB 10138
Laureth-9, Uniqema, Belgium Lot 1127412
Sodium Glycodeoxycholate Prodotti Chimici E Alimentari, Italy Lot 2005010018
Beta-Cyclosporin hydrate, Aldrich Lot 02411 HX
Alfa-Cyclodextrin-(Cavamax W6 Pharma), ISP Lot 60P304
Beta-Cyclodextrin-(Cavamax W7 Pharma), ISP Lot 70P244
Gamma-Cyclodextrin-(Cavamax W7 Pharma), ISP Lot 80P20201
Menthol, MERCK Lot K32726695
Peppermint oil, Frutarom, Lot PPE1504
M-Cresol, Hedinger, Germany, Lot 024015-1
Acesulfame Potassium, Nutrinova, Germany Lot 0000011531
Ethanol, GADOT, Israel, Lot 830109472150

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

1. A solid composition for intra-oral delivery of insulin, comprising; insulin; a hydrophilic polymer matrix; and a phospholipid, providing insulin bioavailability of at least 2%.

2. The solid composition of claim 1 further providing insulin bioavailability of at least 5%.

3. The solid composition of claim 2 further providing insulin bioavailability of at least 10%.

4. The solid composition of claim 3 further providing insulin bioavailability of at least 15%.

5. The solid composition of claim 4 further providing insulin bioavailability of at least 20%.

6. A solid composition for intra-oral delivery of insulin, comprising; insulin; a hydrophilic polymer matrix; and a liposome forming agent, wherein the composition achieves a bioavailability of insulin of at least 2%.

7. The solid composition of claim 6 wherein the composition achieves a bioavailability of insulin of at least 5%.

8. The solid composition wherein the composition achieves a bioavailability of insulin of at least 10%.

9. A solid composition for intra-oral administration of insulin, comprising; Insulin, a hydrophilic polymer matrix, and a phospholipid; wherein upon contact with the oral cavity liquid, said composition forms in-situ particles selected from the group consisting of micelles, emulsions, liposomes, or mixed structures thereof.

10. The solid composition according to claim 9
wherein upon contact with the oral cavity liquid, said composition forms in-situ particles that enhance the absorption of insulin selected from the group consisting of: micelles, emulsions, liposomes and/or mixed structures thereof.

11. The solid composition according to claim 1 adapted for absorption of insulin via gingival, buccal mucosa, lingual mucosa and/or sublingual mucosa.

12. The solid composition according to claim 1 adapted for intra-oral absorption of insulin via gingival buccal mucosa, lingual mucosa and/or sublingual mucosa.

13. The solid composition according to claim 1 wherein the liposome forming agent is select from the group consisting of egg phosphatidylcholine (PC), dilauryl phosphatidylcholine (DLPC), dimyristoyl phosphatidylcholine (DMPC), dipalmitoyl phosphatidylcholine (DPPC), dioleoyl phosphatidylcholine (DOPC), dimyristoyl phosphatidylglycerol (DMPG), dipalmitoyl phosphatidylglycerol(DPPG), dimyristoyl phosphatidic acid(DMPA), dipalmitoyl phosphatidic acid (DPPA), dipalmitoyl phosphatidylethanolamine (DPPE), distearoyl phosphatidylcholine (DSPC), brain phosphatidylserine (PS), brain sphingomyelin (SM), cholesterol(C), cardiolipin (CL), trioctanoin (TC), triolein (TO), soy phosphatidylcholine, poly(adenylic acid), phosphatidylethanolamine (PE), phosphatidyl glycerol (PG), phosphatidyl inositol (PI), sphingosine, cerebroside (glycolipid), and/or the combinations thereof.

14. The solid composition according to claim 1 wherein said composition further contains at least one, stabilizer, preservative, absorption enhancer, antioxidant, chelating agent, sequestrate, antifungal, antimicrobial agent, lubricants, bioadhesive agent, plasticizers, antisticking agents, natural and synthetic flavorings and natural and synthetic colorants, protease inhibitors, wetting agent, suspending agent, surfactant, dispersing agent, buffering agent.

15. The solid composition according to claim 1, wherein the said hydrophilic polymer is selected from the group consisting of Povidone (PVP: polyvinyl pyrrolidone), polyvinyl alcohol, copolymer of PVP and polyvinyl acetate, HPC (hydroxypropyl cellulose), HPMC (hydroxypropyl methylcellulose), carboxymethyl cellulose, hydroxyethyl cellulose, hydroxy Imethyl cellulose, methylcellulose, gelatin, proteins, collagen, hydrolyzed gelatin, polyethylene oxide, acacia, dextrin, magnesium aluminum silicate, starch, a water soluble synthetic polymer, polyacrylic acid, polyhydroxyethylmethacrylate (PHEMA), polyacrylamid, polymethacrylates and their copolymers, gum, water soluble gum, polysaccharide, hydroxypropylmethyl cellulose phthalate, polyvinyl acetate phthalate, cellulose acetate phthalate, hydroxypropylmethyl cellulose acetate succinate, poly(methacrylic acid, methyl methacrylate) 1:1 and poly(methacrylic acid, ethyl acrylate)1:1 , alginic acid, sodium alginate, gums include, for example and without limitation, heteropolysaccharides such as xanthan gum(s), homopolysaccharides such as locust bean gum, galactans, mannans, vegetable gums such as alginates, gum karaya, pectin, agar, tragacanth, accacia, carrageenan, tragacanth, chitosan, agar, alginic acid, other polysaccharide gums (e.g. hydrocolloids), acacia catechu, salai guggal, indian bodellum, copaiba gum, asafetida, cambi gum, Enterolobium cyclocarpum, mastic gum, benzoin gum, sandarac, gambier gum, butea frondosa (Flame of Forest Gum), myrrh, konjak mannan, guar gum, welan gum, gellan gum, tara gum, locust bean gum, carageenan gum, glucomannan, galactan gum, sodium alginate, tragacanth, chitosan, xanthan gum, deacetylated xanthan gum, pectin, sodium polypectate, gluten, karaya gum, tamarind gum, ghatti gum, Accaroid/Yacca/Red gum, dammar gum, juniper gum, ester gum, ipil-ipil seed gum, gum talha (acacia seyal), and cultured plant cell gums including those of the plants of the genera: acacia, actinidia, aptenia, carbobrotus, chickorium, cucumis, glycine, hibiscus, hordeum, letuca, lycopersicon, malus, medicago, mesembryanthemum, oryza, panicum, phalaris, phleum, poliathus, polycarbophil, sida, solanum, trifolium, trigonella, Afzelia africana seed gum, Treculia africana gum, detarium gum, cassia gum, carob gum, Prosopis africana gum, Colocassia esulenta gum, Hakea gibbosa gum, khaya gum, scleroglucan, zea, a water insoluble cross-linked polysaccharide, a water insoluble polysaccharide, a water insoluble synthetic polymer, a water insoluble cross-linked protein, a water insoluble cross-linked peptide, water insoluble cross-linked gelatin, water insoluble cross-linked hydrolyzed gelatin, water insoluble cross-linked collagen, water insoluble cross linked polyacrylic acid, water insoluble cross- linked cellulose derivatives, water insoluble cross-linked polyvinyl pyrrolidone, micro crystalline cellulose, insoluble starch, micro crystalline starch and a combination thereof, insoluble metal salts or cross-linked derivatives of alginate, pectin, xantham gum, guar gum, tragacanth gum, locust bean gum, carrageenan, and metal salts thereof, and covalently cross-linked derivatives thereof, cross-linked derivatives of hydroxypropylcellulose, hydroxypropylmethylcellulose, hydroxyethylcellulose, methylcellulose, hydroxymethyl cellulose, carboxymethylcellulose, and metal salts of carboxymethylcellulose, mixtures of any of the foregoing, and the like and any other pharmaceutically acceptable polymer that dissolves in buffer phosphate pH>5.5 and/or mixtures thereof.

16. A solid composition for intra-oral delivery comprising; a pharmaceutically acceptable active agent; a hydrophilic polymer matrix; and a phospholipid, wherein the composition provides bioavailability of said pharmaceutically acceptable active agent of at least about 5% and said pharmaceutically acceptable active agent has a dissolution rate higher than that of the said hydrophilic polymer.

17. A solid composition for intra-oral delivery comprising; a pharmaceutically acceptable active agent; a hydrophilic polymer matrix; and a phospholipid, wherein the composition provides bioavailability of said pharmaceutically acceptable active agent of at least about 5% and said pharmaceutically acceptable active agent has a dissolution rate higher than that of any excipient present in the matrix including the phospholipids or mixture thereof.

18. A solid composition for intra-oral delivery of insulin comprising; insulin, a hydrophilic polymer matrix and a phospholipid providing a reduction of blood glucose levels of a subject by at least 5%.

19. A solid composition comprising a hydrophilic polymer matrix, at least one phospholipid and insulin.

20. The solid composition of claim 19 comprising a hydrophilic polymer matrix, lecithin and insulin providing the reduction of glucose blood level of a subject by at least about 5%.

21. The solid composition of claim 19 comprising a hydrophilic polymer matrix, phosphotidylcholine and insulin providing the reduction of glucose blood level of a subject by at least about 5%.

22. A solid composition according to claim 1 that provides a reduction of blood glucose levels of a subject by at least about 5%.

23. The method for the reduction of the blood glucose plasma levels of the subject by at least 5% comprising administering to said subject a solid composition of claim 19.

24. The method for treating Type I diabetes comprising the intra-oral use of solid composition of claim 19.

25. The method for decreasing the need for at least one subcutaneous injection a day for Type I diabetes patients comprising the intra-oral use of the solid composition comprising: insulin, a hydrophilic polymer matrix and a phospholipid.

26. The method for treating Type II diabetes comprising the intra-oral use of a solid composition of claim 19.

27. The method for decreasing the need for at least one subcutaneous injection a day for Type II diabetes patients comprising the intra-oral use of the solid composition of claim 19.
